SEQUENCE LISTING (42 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 22 nt, Homo sapiens
ugagguaguaguuuguacaguu

SEQ ID NO: 8 - RNA, 22 nt, Homo sapiens
ugagguaguaguuugugcuguu

SEQ ID NO: 9 - RNA, 24 nt, Homo sapiens
ucccugagacccuuuaaccuguga

SEQ ID NO: 10 - RNA, 22 nt, Homo sapiens
ucccugagacccuaacuuguga

SEQ ID NO: 11 - RNA, 22 nt, Homo sapiens
uagcagcacaucaugguuuaca

SEQ ID NO: 12 - RNA, 22 nt, Homo sapiens
uagcagcacguaaauauuggcg

SEQ ID NO: 13 - RNA, 23 nt, Homo sapiens
cccaguguucagacuaccuguuc

SEQ ID NO: 14 - RNA, 22 nt, Homo sapiens
uagcuuaucagacugauguuga

SEQ ID NO: 15 - RNA, 22 nt, Homo sapiens
uucccuuugucauccuucgccu

SEQ ID NO: 16 - RNA, 22 nt, Homo sapiens
acagcaggcacagacaggcagu

SEQ ID NO: 17 - RNA, 23 nt, Homo sapiens
agcuacauugucugcuggguuuc

SEQ ID NO: 18 - RNA, 21 nt, Homo sapiens
agcuacaucuggcuacugggu

SEQ ID NO: 19 - RNA, 21 nt, Homo sapiens
aucacauugccagggauuucc

SEQ ID NO: 20 - RNA, 21 nt, Homo sapiens
aucacauugccagggauuacc

SEQ ID NO: 21 - RNA, 22 nt, Homo sapiens
ccuauucuugguuacuugcacg

SEQ ID NO: 22 - RNA, 23 nt, Homo sapiens
uguaaacauccuacacucucagc

SEQ ID NO: 23 - RNA, 22 nt, Homo sapiens
aaaagcuggguugagagggcga

SEQ ID NO: 24 - RNA, 22 nt, Homo sapiens
aacccguagauccgaucuugug

SEQ ID NO: 25 - DNA, 47 bp, Homo sapiens
accacaattaattgtaattaagggaaatgaattattaaaacaatttt

SEQ ID NO: 26 - DNA, 47 bp, Artificial Sequence
  Description of Artificial Sequence Synthetic oligonucleotide
accacaattaattgtaatttacgcatatgaattattaaaacaatttt

SEQ ID NO: 27 - DNA, 22 bp, Artificial Sequence
  Description of Artificial Sequence Synthetic oligonucleotide
ttccctttgtcatccttcgcct

SEQ ID NO: 28 - DNA, 22 bp, Artificial Sequence
  Description of Artificial Sequence Synthetic oligonucleotide
aggcgaaggatgacaaagggaa

SEQ ID NO: 29 - DNA, 55 bp, Artificial Sequence
  Description of Artificial Sequence Synthetic oligonucleotide
cgtacttcaatgacaatatttcaagagaatattgtcattgaagtacgtctttttt

SEQ ID NO: 30 - DNA, 55 bp, Artificial Sequence
  Description of Artificial Sequence Synthetic oligonucleotide
aaaaaagacgtacttcaatgacaatattctcttgaaatattgtcattgaagtacg

SEQ ID NO: 31 - DNA, 37 bp, Artificial Sequence
  Description of Artificial Sequence Synthetic primer
tgcggccgccttccctatatctaaacaatgcaaaatc

SEQ ID NO: 32 - DNA, 27 bp, Artificial Sequence
  Description of Artificial Sequence Synthetic primer
aaccggtcacccatccaggcgaggagc

SEQ ID NO: 33 - DNA, 28 bp, Artificial Sequence
  Description of Artificial Sequence Synthetic primer
tacgcatatgaattattaaaacaatttt

SEQ ID NO: 34 - DNA, 27 bp, Artificial Sequence
  Description of Artificial Sequence Synthetic primer
tatgcgtaaattacaattaattgtgct

SEQ ID NO: 35 - DNA, 10 bp, Artificial Sequence
  Description of Artificial Sequence Synthetic oligonucleotide
gctcacatgt

SEQ ID NO: 36 - DNA, 749 bp, Homo sapiens
tagatctctcagatgctggggaagacccagcccctctgcagagagggatagttcagggtttgggttttttctttcctcctgggctgagaaagctcatggaaagctggaataaccacgcatactgttacagcaaatccaacagagctcccaccgtggtggttttcaggtgactgtggatgccaagcaggcaagctcctggtgaaggggggagagcagggattagaagcactcagaaggggctgagagtcatgtggggctcacactgcatttgcagctgggttcaccctgacctcaggcccaacttagatgaggaaggattagcagtaattagtgccatgtgccgccttctcccagctccccggggcacgaacactgcccagctgatgaggggattctgaaagaaccattatgtccaattgtctcaattatgcaaaccctgctgacatttccagccagggaagggcggctgggtgggagggggccatggcggggccacttcaaaggaaaagctctagctcccctacctctctcacatcctaaggctgcctttgtgggattccacacagaacagcctggaagcttggggccctggcttccttttctggcctgggagtcaggtcatggggccatcgcttcacagcaatcatgagggcccaggcccaagtgctcacatgctcctcatggggactgctcctcttaaagggtgggccctcctcacccagctccctgccctggccaaggagctagct

SEQ ID NO: 37 - DNA, 749 bp, Homo sapiens
tagatctctcagatgctggggaagacccagcccctctgcagagagggatagttcagggtttgggttttttctttcctcctgggctgagaaagctcatggaaagctggaataaccacgcatactgttacagcaaatccaacagagctcccaccgtggtggttttcaggtgactgtggatgccaagcaggcaagctcctggtgaaggggggagagcagggattagaagcactcagaaggggctgagagtcatgtggggctcacactgcatttgcagctgggttcaccctgacctcaggcccaacttagatgaggaaggattagcagtaattagtgccatgtgccgccttctcccagctccccggggcacgaacactgcccagctgatgaggggattctgaaagaaccattatgtccaattgtctcaattatgcaaaccctgctgacatttccagccagggaagggcggctgggtgggagggggccatggcggggccacttcaaaggaaaagctctagctcccctacctctctcacatcctaaggctgcctttgtggagggggccatggcggggccacttcaaaggaaaagctctagctcccctacctctctcacatcctaaggctgcctttgtgcttcacagcaatcatgagggcccaggcccaagtgctcacatgctcctcatggggactgctcctcttaaagggtgggccctcctcacccagctccctgccctggccaaggagctagct

SEQ ID NO: 38 - DNA, 749 bp, Homo sapiens
tagatctctcagatgctggggaagacccagcccctctgcagagagggatagttcagggtttgggttttttctttcctcctgggctgagaaagctcatggaaagctggaataaccatgcatactgttacagcaaatccaacagagctcccaccgtggtggttttcaggtgactgtggatgccaagcaggcaagctcctggtgaaggggggagagcagggattagaagcactcagaaggggctgagagtcatgtggggctcacactgcatttgcagctgggttcaccctgacctcaggcccaacttagatgaggaaggattagcagtaattaatgccatgtgccgccttctcccagctccccggggcacgaacactgcccagctgatgaggggattctgaaagaaccattatgtccaattgtctcaattatgcaaaccctgctgacatttccagccagggaagggcggctgggtgggagggggccatggcggggccacttcaaaggaaaagctctagctcccctacctctctcacatcctaaggctgcctttgtggagggggccatggcggggccacttcaaaggaaaagctctagctcccctacctctctcacatcctaaggctgcctttgtgcttcacagcaatcatgagggcccaggcccaagtgctcacatgctcctcatggggactgctcctcttaaagggtggaccctcctcacccagctccctgccctggccaaggagctagct

SEQ ID NO: 39 - DNA, 749 bp, Homo sapiens
tagatctctcagatgctggggaagacccagcccctctgcagagagggatagttcagggttcgggttttttctttcctcctgggctgagaaagctcatggaaagctggaataaccatgcatactgttacagcaaatccaacagggctcccaccatggtggttttcaggtgactgtggatgccaagcaggcaagctcctggtgaaggggggagagcagggattagaagcactcagaaggggctgagagtcatgtggggctcacactgcatttgcagctgggttcaccctgacctcaggcccaacttagatgaggaaggattagcagtaattaatgccatgtgccgccttctcccagctccccggggcacgaacactgcccagctgatgaggggattctgaaagaaccattatgtccaattgtctcaattatgcaaaccctgctgacatttccagccagggaagggcggctgggtgggagggggccatggcggggccacttcaaaggaaaagctctagctcccctacctctctcacatcctaaggctgcctttgtggagggggccatggcggggccacttcaaaggaaaagctctagctcccctacctctctcacatcctaaggctgcctttgtgcttcacagcaatcatgagggcccaggcccaagtgctcacatgctcctcatggggactgctcctcttaaagggtggaccctcctcacccagccccctgccctggccaaggagctagct

SEQ ID NO: 40 - DNA, 8 bp, Homo sapiens
aaagggaa

SEQ ID NO: 41 - DNA, 4648 bp, Homo sapiens
tgatgcgtccccccccaacctttccctcaccccctcccacccccagccccgactccagccagcgcctccctccaccccaggacgccactcatttcatctcatttaagggaaaaatatatatctatctatttgaggaaactgaggacctcggaatctctagcaagggctcaacttcgaaaatggcaacaacagagatgcaaaaagctaaaaagacaccccccccctttaaatggttttctttttgaggcaagttggatgaacagagaagggaagagaggaagaacgagaggaagagaagggaaggaagtgtttgtgtagaagagagagaaagacgaatagagttaggaaaaggaagacaagcaggtgggcaggaaggacatgcaccgagaccaggcaggggcccaactttcacgtccagccctggcctggggtcgggagaggtgggcgctagaagatgcagcccaggatgtggcaatcaatgacactattggggtttcccaggatggattggtcagggggagaaaggaaaaggcaaaacactccaggacctctcccggatctgtctcctcctctagccagcagtatggacagctggacccctgaacttcctctcctcttacctgggcagagtgttgtctctccccaaatttataaaaactaaaatgcattccattcctctgaaagcaaaacaaattcataattgagtgatattaaatagagaggttttcggaagcagatctgtgaatatgaaatacatgtgcatatttcattccccaggcagacattttttagaaatcaatacatgccccaatattggaaagacttgttcttccacggtgactacagtacatgctgaagcgtgccgtttcagccctcatttaattcaatttgtaagtagcgcagcagcctctgtgggggaggataggctgaaaaaaaaaagtgggctcgtatttatctacaggactccatatagtcatatataggcatataaatctattctttttctttgtttttttctttcttcctttctttcaaaggtttgcattaacttttcaaagtagttcctataggggcattgaggagcttcctcattctgggaaaactgagaaaacccatattctcctaatacaacccgtaatagcatttttgcctgcctcgaggcagagtttcccgtgagcaataaactcagcttttttgtggggcacagtactggatttgacagtgattccccacgtgtgttcatctgcacccaccgagccaggcagaggccagccctccgtggtgcacacagcacgcgcctcagtccatcccattttagtctttaaaccctcaggaagtcacagtctccggacaccacaccacatgagcccaacaggtccacgatggatccaccagtcccaccccagccttttcctttcatctgaacagaatgtgcatttttggaagcctccctcactctccatgctggcagagcaggagggagactgaagtaagagatggcagagggagatggtggcaaaaaggtttagatgcaggagaacagtaagatggatggttccggccagagtcgatgtggggaggaacagagggctgaagggagagggggctgactgttccattctagctttggcacaaagcagcagaaagggggaaaagccaatagaaatttccttagcttccccaccatatgtattttctaggatttgagaggaaagagaggaaaatgggggaatgggttgcaaaatagaaatgagcttaatccaggccgcagagccagggaaggtgagtaactttaggagggtgctagactttagaagccagataggaagaatcagtctaaactggccatgctttggaagggacaagactatgtgctccgctgcccaccttcagcctgcaatgagggactgaggcccacgagtctttccagctcttcctccattctggccagtccctgcatcctccctggggtggaggatggaaggaaagctgggacaagcagggaacgcatgattcagggatgctgtcactcggcagccagattccgaaactcccattctccaatgacttcctcaaccaatgggtggccttgtgactgttctttaaggctgaagatatccaggaaagggggcttggacactggccaaggagaccccttcgtgctgtggacacagctctcttcactctttgctcatggcatgacacagcggagaccgcctccaacaacgaatttggggctacgaagaggaatagcgaaaaagcaaatctgtttcaactgatgggaaccctatagctatagaacttgggggctatctcctatgcccctggacaggacagttggctggggacaggagaagtgctcaatcttcatgagacaaaggggcccgatagggccagcagccacaaggccttgacctgccgagtcagcatgccccatctctctgcacagctgtcccctaaacccaactcacgtttctgtatgtcttaggccagtatcccaaacctcttccacgtcactgttctttccacccattctccctttgcatcttgagcagttatccaactaggatctgccaagtggatactggggtgccactcccctgagaaaagactgagccaggaactacaagctccccccacattcctcccagcctggacctaattcttgagaggggctctctcttcacggactgtgtctggactttgagcaggcttctgccccttgcgttggctctttgctgccagccatcaggtgggggattagagcctggtgtaagtgcgccagactcttccggtttccaaagttcgtgcctgcgaacccaaacctgtgagtctcttctgcatgcaggagtttctcctgggcagctggtcactccccagagaagctgggccttcatggacacatggaactaagcctcccaaatgggagttctggctgagcccagggtggggagatcctgggaagggaggcactggaggaagacggcacctcttcccccatggcagggtgtgagggaggcaggtttggaatggtgcgagtatggcaatctaagcaggggtctggtctctttgactccaggctggcctttggccgactgtctgctcacccagagaccttggactccggactatccatggctccgaatctaagtgctgcccactcccatgctcacacccacagaaggtcttcccatcccctttagattcgtgcctcactccaccagtgaggaagatgcctctgtctttcccacgactgccaggagatagggaagcccagccaggactgaccctccttcctccagcctgccctgacccacctggcaaagcagggcacatggggaggaagagactggaacctttctttgacagccaggcctagacagacaggcctggggacactggccccatgaggggaggaaggcaggcgcacgaggtccagggaggcccttttctgatcatgccccttctctcccaccccatctccccaccaccacctctgtggcctccatggtacccccacagggctggcctcccctagagggtgggcctcaaccacctgctcccgccacgcaccggttagtgagacagggctgccacggcaaccgccaagcccccctcaaggtgggacagtaccccggacccatccactcactcctgagagggctccggcccagaatgggaacctcagagaagagctctaaggagaagaaaccccatagcgtcagagaggatatgtctggcttccaagagaaaggaggctccgttttgcaaagtggaggagggacgagggacaggggtttcaccagccagcaacctgggccttgtactgtctgtgtttttaaaaccactaaagtgcaagaattacattgcactgtttctccactttttattttctcttaggcttttgtttctatttcaaacatactttcttggttttctaatggagtatatagtttagtcatttcacagactctggcctcctctcctgaaatccttttggatggggaaagggaaggtggggagggtccgaggggaaggggaccccagcttccctgtgcccgctcaccccactccaccagtccccggtcgccagccggagtctcctctctaccgccactgtcacaccgtagcccacatggatagcacagttgtcagacaagattccttcagattccgagttgcctaccggttgttttcgttgttgttgttgttgtttttctttttctttttttttttgaagacagcaataaccacagtacatattactgtagttctctatagttttacatacattcataccataactctgttctctcctcttttttgttttcaactttaaaaacaaaaataaacgatgataatctttactggtgaaaaggatggaaaaataaatcaacaaatgcaaccagtttgtgagaaaaaaaaaaaaaagccgaaaaaaaaaaaaaaaacacctgaatgcggaagagctcggctcccgtttagcattttgtacttaaggaaataaaaaaccaacaaaggatctcacattttcttaaaaagtgaagattgctgtatactatttattcaacttataatttatgttactccttgatctttgtcttttgtcatgacaaagcatttatttaataaagttatgcattcagtt

SEQ ID NO: 42 - DNA, 8 bp, Artificial Sequence
  Description of Artificial Sequence Synthetic oligonucleotide
taagccta

(19) United States
(12) Patent Application Publication
(10) Pub. No.: US 2012/0108457 A1
(43) Pub. Date: May 3, 2012

(54) MIR-211 EXPRESSION AND RELATED PATHWAYS IN HUMAN MELANOMA

(75) Inventors: Ranjan Perera, Orlando, FL (US); Joseph Mazar, Orlando, FL (US)

(73) Assignee: Sanford-Burnham Medical Research Institute

(21) Appl. No.: 13/271,030

(22) Filed: Oct. 11, 2011

Related U.S. Application Data
(60) Provisional application No. 61/442,108, filed on Feb. 11, 2011.
(60) Provisional application No. 61/391,948, filed on Oct. 11, 2010.

(51) Int. Cl. C40B 30/04; C12Q 1/68

(52) U.S. Cl. 506/9; 435/6.14; 435/6.12

(57) ABSTRACT

Provided herein are methods for the diagnosis of human melanoma by assessing MITF, miR-211, TRPM1, and/or KCNMA1. Methods for treating melanoma are also provided.

### CROSS REFERENCE TO RELATED APPLICATIONS

This application claims benefit of U.S. Provisional Patent Application Nos. 61/391,948, filed Oct. 11, 2010 and 61/442,108, filed Feb. 11, 2011, each of which is incorporated by reference in its entirety.

### STATEMENT OF GOVERNMENT-SPONSORED RESEARCH

This invention was made with United States government support awarded by the following agencies: National Institutes of Health under Grant No. 1R01GM084881-01, and the National Science Foundation under Grant No. FIBR 0527023. The United States government has certain rights in the invention.

### FIELD OF THE INVENTION

The present invention relates to methods of diagnosing and treating human melanoma.

### BACKGROUND OF THE INVENTION

The following discussion of the background of the invention is merely provided to aid the reader in understanding the invention and is not admitted to describe or constitute prior art to the present invention.

Melanoma, a cancer of the pigment-producing cells in the skin epidermis, can be highly metastatic, and malignant melanomas are relatively resistant to standard chemotherapy. A major cause for melanoma initiation is extensive or intermittent exposure to the sun's radiation over a period of time, and the extent of melanin pigmentation is an important risk factor. The exact molecular mechanisms that lead to melanoma are complex and poorly understood, and may involve both mutagenic DNA lesions and epigenetic misregulation. The complexity is added by the involvement of several different signal transduction pathways, such as the Hedgehog pathway, which controls BCL2-mediated apoptosis; mutations in the Patched gene, the endpoint of the Hedgehog pathway, have also been correlated with skin cancers [3, 12-15].

A frequent causative mechanism for an inherited form of predisposition to melanoma is thought to be a chromosomal deletion over 9p21. The 9p21 site harbors the tumor suppressor gene INK4a and accompanies additional inactivating mutations that lead to the constitutive activation of genes such as BRAF [16, 17]. INK4a encodes one of several cyclin-dependent protein kinase inhibitors, which is located adjacent to an alternate reading frame of the human p14^{ARF}. P14^{ARF} binds to the Mdm2 protein in several cell lines (though remains untested in melanoma cell lines, to our knowledge) and thereby abrogates Mdm2's binding to p53, causing p53 to be stabilized and nuclear localized. The loss of INK4a therefore may lead to interference of two separate pathways of cell cycle control: CDK signaling and suppression of p53 activity by Mdm2-induced acceleration of p53 degradation. Methylation near the 5′ upstream region of INK4a has been shown in some 10% of melanomas [7], suggesting that epigenetic down-regulation of this gene may be important for melanoma development. The activation of BRAE alone may be insufficient to cause metastatic melanoma, but additional mutagenic or epigenetic events such as the inactivation of tumor suppressor genes, e.g., Pten [18], may be important. There is evidence that the NOTCH signaling pathway is also important for distinguishing normal melanocytes from melanoma cells [19, 20].

Measurement of genome-wide DNA copy number variations, together with analysis of somatic mutations in specific marker genes, can be used to distinguish among different melanoma subtypes with reasonable accuracy [21]. Particularly noteworthy is the recent demonstration of abnormally high oncogenic potentials of single melanoma cells [22], emphasizing the need for a better understanding the molecular mechanisms of melanoma progression.

In the search for such an understanding, attention has recently focused on the role of small non-coding RNA molecules in cancer development [23-27] and in melanoma in particular [28-32]. miRNAs influence cancer development by serving either as tumor suppressors or oncogenes [33-39] by their negative regulatory effects on mRNA encoded by oncogenes or tumor suppressor genes, respectively. With the goal of defining the genes with major contributions to melanoma, several genome-wide expression level studies have identified a number of protein-coding [40] and microRNA (miRNA) genes as important players [32, 41-43]. Several of these genes and their expression signatures exhibit distinct patterns among malignant metastatic melanomas and their benign forms, but their significance with respect to melanoma initiation and progression is poorly understood. For example, miR-221/222 were found to down-regulate p27Kip1/CDKN1B and the c-KIT receptor, which controls the progression of neoplasia leading to enhanced proliferation and reduced differentiation in melanoma cells [42]. Similarly, high miR-137 expression in melanoma cell lines down-regulates microphthalma associated transcription factor (MITF), a transcription factor important for melanocyte cell growth, maturation, apoptosis, and pigmentation [32]. The depletion of miR-182 reduces invasiveness and induces melanoma cell death by suppressing the expression of transcription factors FOXO3 and MITF [43], suggesting that its increased expression may be associated with certain aspects of melanoma biology.

### SUMMARY OF THE INVENTION

The present invention is based on the discovery of the correlation between miRNA-211 expression and regulation and human melanoma.

In a first aspect, the present invention provides a method for diagnosing melanoma in a subject suspected of having melanoma comprising: (i) assessing the expression level of KCNMA1 in a biological sample obtained from the subject; (ii) comparing the expression level of KCNMA1 in the sample to a reference expression level derived from the expression level of KCNMA1 in samples obtained from subjects diagnosed as not having melanoma; and (iii) identifying the subject as having melanoma when the expression level of KCNMA1 in the sample is greater than the reference expression level or identifying the subject as not having melanoma when the expression level of KCNMA1 in the sample is not greater than the reference expression level. In some embodiments, the biological sample may comprise skin, skin epidermis, or melanocytes.

In further embodiments, the expression level of KCNMA1 may be assessed by evaluating the amount of KCNMA1 mRNA in the biological sample. Such an evaluation of the amount of KCNMA1 mRNA may comprise reverse transcriptase PCR (RT-PCR), or, in further embodiments, may comprise array hybridization, wherein the array comprises an immobilized nucleic acid probe that specifically hybridizes KCNMA1 mRNA, KCNMA1 cDNA, or complements thereof. In still further embodiments, the expression level of KCNMA1 is assessed by evaluating the amount of KCNMA1 protein in the biological sample.

Another aspect of the present invention provides a method for determining the risk of a subject for developing melanoma comprising: (i) assessing the expression level of KCNMA1 in a biological sample obtained from the subject; (ii) comparing the expression level of KCNMA1 in the sample to the a reference expression level derived from the expression level of KCNMA1 in samples obtained from subjects diagnosed as not having melanoma; and (iii) identifying the subject as having increased risk of developing melanoma when the expression level of KCNMA1 in the sample is greater than the reference expression level or identifying the subject as not having an increased risk of melanoma when the expression level of KCNMA1 in the sample is not greater than the reference expression level. In some embodiments, the biological sample may comprise skin, skin epidermis, or melanocytes.

In further embodiments, the expression level of KCNMA1 may be assessed by evaluating the amount of KCNMA1 mRNA in the biological sample. Such an evaluation of the amount of KCNMA1 mRNA may comprise reverse transcriptase PCR (RT-PCR), or, in further embodiments, may comprise array hybridization, wherein the array comprises an immobilized nucleic acid probe that specifically hybridizes KCNMA1 mRNA, KCNMA1 cDNA, or complements thereof. In still further embodiments, the expression level of KCNMA1 is assessed by evaluating the amount of KCNMA1 protein in the biological sample.

In another aspect, the present invention provides a method for treating a patient diagnosed as having melanoma comprising administering to the patient an effective amount of a therapeutic agent that reduces KCNMA1 biological activity. The biological activity may, in some embodiments, be reduced in the melanoma cells by, in further embodiments, at least 10%, at least 50%, or at least 90%.

In some embodiments, the therapeutic agent may comprise a KCNMA1 siRNA, a KCNMA1 anti-sense nucleic acid, an anti-KCNMA1 antibody, or a nucleic acid encoding miR-211. Such a nucleic acid may also be encoded in a vector or a viral vector. Additionally, the therapeutic agent may be contained within a liposome in some embodiments. In some embodiments, it may reduce the expression of KCNMA1 mRNA or KCNMA1 protein, or inhibit the potassium conductance of the KCNMA1 protein.

In still another aspect, the present invention provides a method for determining the proliferation rate of melanoma in a subject comprising: (i) assessing the expression level of KCNMA1 in a melanoma sample obtained from the subject; and (ii) identifying the proliferation rate of the melanoma, wherein a higher expression level of KCNMA1 in the sample indicates a greater proliferation rate and a lower expression level of KCNMA1 in the sample indicates a lower proliferation rate.

In further embodiments, the expression level of KCNMA1 may be assessed by evaluating the amount of KCNMA1 mRNA in the melanoma sample. Such an evaluation of the amount of KCNMA1 mRNA may comprise reverse transcriptase PCR (RT-PCR), or, in further embodiments, may comprise array hybridization, wherein the array comprises an immobilized nucleic acid probe that specifically hybridizes KCNMA1 mRNA, KCNMA1 cDNA, or complements thereof. The expression level of KCNMA1 may also, in some embodiments, be assessed by evaluating the amount of KCNMA1 protein in the melanoma sample.

In yet another aspect of the present invention, a method is provided for determining the metastatic potential of melanoma in a subject comprising: (i) assessing the expression level of KCNMA1 in a melanoma sample obtained from the subject; and (ii) identifying the metastatic potential of the melanoma, wherein a higher expression level of KCNMA1 in the sample indicates a greater metastatic potential and a lower expression level of KCNMA1 in the sample indicates a lower metastatic potential.

In further embodiments, the expression level of KCNMA1 may be assessed by evaluating the amount of KCNMA1 mRNA in the melanoma sample. Such an evaluation of the amount of KCNMA1 mRNA may comprise reverse transcriptase PCR (RT-PCR), or, in further embodiments, may comprise array hybridization, wherein the array comprises an immobilized nucleic acid probe that specifically hybridizes KCNMA1 mRNA, KCNMA1 cDNA, or complements thereof. The expression level of KCNMA1 may also, in some embodiments, be assessed by evaluating the amount of KCNMA1 protein in the melanoma sample.

In another aspect, the present invention provides a method for diagnosing melanoma in a subject suspected of having melanoma comprising: (i) assessing the expression level of MITF in a biological sample obtained from the subject; (ii) comparing the expression level of MITF in the sample to the a reference expression level derived from the expression level of WIT in samples obtained from subjects diagnosed as not having melanoma; and (iii) identifying the subject as having melanoma when the expression level of MITF in the sample is less than the reference expression level or identifying the subject as not having melanoma when the expression level of MITF in the sample is not less than the reference expression level. In some embodiments, the biological sample may comprise skin, skin epidermis, or melanocytes.

In some embodiments, the expression level of MITF is assessed by evaluating the amount of MITF mRNA in the biological sample. Such evaluation may, in some embodiments, comprise reverse transcriptase PCR (RT-PCR). In further embodiments, such evaluation may comprise array hybridization, wherein the array comprises an immobilized nucleic acid probe that specifically hybridizes MITF mRNA, MITF cDNA, or complements thereof, expression level of MITF may additionally be assessed by evaluating the amount of MITF protein in the biological sample.

In still a further aspect of the present invention, a method is provided for determining the risk of a subject for developing melanoma comprising: (i) assessing the expression level of MITF in a biological sample obtained from the subject; (ii) comparing the expression level of MITF in the sample to the a reference expression level derived from the expression level of MITF in samples obtained from subjects diagnosed as not having melanoma; and (iii) identifying the subject as having increased risk of developing melanoma when the expression level of MITF in the sample is less than the reference expression level or identifying the subject as not having an increased risk of melanoma when the expression level of MITF in the sample is not less than the reference expression level. In some embodiments, the biological sample may comprise skin, skin epidermis, or melanocytes.

In some embodiments, the expression level of MITF is assessed by evaluating the amount of MITF mRNA in the biological sample. Such evaluation may, in some embodiments, comprise reverse transcriptase PCR (RT-PCR). In further embodiments, such evaluation may comprise array hybridization, wherein the array comprises an immobilized nucleic acid probe that specifically hybridizes MITF mRNA, MITF cDNA, or complements thereof, expression level of MITF may additionally be assessed by evaluating the amount of MITF protein in the biological sample.

In yet another aspect, the present invention provides a method for treating a patient diagnosed as having melanoma comprising administering to the patient an effective amount of a therapeutic agent that increases MITF biological activity. In some embodiments, the MITF biological activity is increased in the melanoma cells by, in further embodiments, at least 10%, at least 50%, or at least 100%.

In some embodiments, the therapeutic agent may comprise a nucleic acid encoding MITF. Such a nucleic acid may, in some embodiments, be encoded in a vector or a viral vector. The therapeutic agent may additionally be contained within a liposome. The administration of the therapeutic agent may, in further embodiments, result in an increase in the expression of miR-211 or TRPM1, or may result in a reduction in the expression of KCNMA1.

In another aspect, the present invention provides a method for determining the proliferation rate of melanoma in a subject comprising: (i) assessing the expression level of MITF in a melanoma sample obtained from the subject; and (ii) identifying the proliferation rate of the melanoma, wherein a lower expression level of MITF in the sample indicates a greater proliferation rate and a higher expression level of MITF in the sample indicates a lower proliferation rate.

In some embodiments, the expression level of MITF is assessed by evaluating the amount of MITF mRNA in the melanoma sample. Such an evaluation may, in further embodiments, comprise reverse transcriptase PCR (RT-PCR) or array hybridization, wherein the array comprises an immobilized nucleic acid probe that specifically hybridizes MITF mRNA, MITF cDNA, or complements thereof. The expression level of MITF may further be assessed by evaluating the amount of MITF protein in the biological sample.

Yet another aspect of the invention provides a method determining the metastatic potential of melanoma in a subject comprising: assessing the expression level of MITF in a melanoma sample obtained from the subject; and (ii) identifying the metastatic potential of the melanoma, wherein a lower expression level of MITF in the sample indicates a greater metastatic potential and a higher expression level of MITF in the sample indicates a lower metastatic potential.

In some embodiments, the expression level of MITF is assessed by evaluating the amount of MITF mRNA in the melanoma sample. Such an evaluation may, in further embodiments, comprise reverse transcriptase PCR (RT-PCR) or array hybridization, wherein the array comprises an immobilized nucleic acid probe that specifically hybridizes MITF mRNA, MITF cDNA, or complements thereof. The expression level of MITF may further be assessed by evaluating the amount of MITF protein in the biological sample.

In still another aspect of the present invention, a method is provided for diagnosing melanoma in a subject suspected of having melanoma comprising: (i) assessing the expression level of TRPM1 in a biological sample obtained from the subject; (ii) comparing the expression level of TRPM1 in the sample to the a reference expression level derived from the expression level of TRPM1 in samples obtained from subjects diagnosed as not having melanoma; and (iii) identifying the subject as having melanoma when the expression level of TRPM1 in the sample is less than the reference expression level or identifying the subject as not having melanoma when the expression level of TRPM1 in the sample is not less than the reference expression level. In some embodiments, the biological sample may comprise skin, skin epidermis, or melanocytes.

In further embodiments, the expression level of TRPM1 is assessed by evaluating the amount of TRPM1 mRNA in the biological sample. Such an evaluation may, in some embodiments, comprise reverse transcriptase PCR (RT-PCR) or, in further embodiments, array hybridization, wherein the array comprises an immobilized nucleic acid probe that specifically hybridizes TRPM1 mRNA, TRPM1 cDNA, or complements thereof. The expression level of TRPM1 may be assessed in further embodiments by evaluating the amount of TRPM1 protein in the biological sample.

In yet another aspect of the present invention, a method is provided for determining the risk of a subject for developing melanoma comprising: assessing the expression level of TRPM1 in a biological sample obtained from the subject; (ii) comparing the expression level of TRPM1 in the sample to the a reference expression level derived from the expression level of TRPM1 in samples obtained from subjects diagnosed as not having melanoma; and (iii) identifying the subject as having increased risk of developing melanoma when the expression level of TRPM1 in the sample is less than the reference expression level or identifying the subject as not having an increased risk of melanoma when the expression level of TRPM1 in the sample is not less than the reference expression level. In some embodiments, the biological sample may comprise skin, skin epidermis, or melanocytes.

In further embodiments, the expression level of TRPM1 is assessed by evaluating the amount of TRPM1 mRNA in the biological sample. Such an evaluation may, in some embodiments, comprise reverse transcriptase PCR (RT-PCR) or, in further embodiments, array hybridization, wherein the array comprises an immobilized nucleic acid probe that specifically hybridizes TRPM1 mRNA, TRPM1 cDNA, or complements thereof. The expression level of TRPM1 may be assessed in further embodiments by evaluating the amount of TRPM1 protein in the biological sample.

In another aspect, the present invention provides a method for treating a patient diagnosed as having melanoma comprising administering to the patient an effective amount of a therapeutic agent that increases TRPM1 biological activity. In some embodiments, the TRPM1 biological activity is increased in the melanoma cells by, in further embodiments, at least 10%, at least 50%, or at least 100%.

In further embodiments, the therapeutic agent may comprise a nucleic acid encoding TRPM1. The nucleic acid may be encoded in a vector or a viral vector, or may be contained within a liposome. The administration of the therapeutic agent may, in some embodiments, result in an increase in the expression of miR-211, or a reduction in the expression of KCNMA1.

In still another aspect of the present invention, a method is provided for determining the proliferation rate of melanoma in a subject comprising: (i) assessing the expression level of TRPM1 in a melanoma sample obtained from the subject; and (ii) identifying the proliferation rate of the melanoma, wherein a lower expression level of TRPM1 in sample indicates a greater proliferation rate and a higher expression level of TRPM1 in the sample indicates a lower proliferation rate.

In some embodiments, the expression level of TRPM1 assessed by evaluating the amount of TRPM1 mRNA in the melanoma sample. Such an evaluation may, in some embodiments, comprise reverse transcriptase PCR (RT-PCR) or, in further embodiments, array hybridization, wherein the array comprises an immobilized nucleic acid probe that specifically hybridizes TRPM1 mRNA, TRPM1 cDNA, or complements thereof. The expression level of TRPM1 may be assessed in further embodiments by evaluating the amount of TRPM1 protein in the melanoma sample.

Yet another aspect of the present invention provides a method for determining the metastatic potential of melanoma in a subject comprising: (i) assessing the expression level of TRPM1 in a melanoma sample obtained from the subject; and (ii) identifying the metastatic potential of the melanoma, wherein a lower expression level of TRPM1 in the sample indicates a greater metastatic potential and a higher expression level of TRPM1 in the sample indicates a lower metastatic potential.

In some embodiments, the expression level of TRPM1 is assessed by evaluating the amount of TRPM1 mRNA in the melanoma sample. Such an evaluation may, in some embodiments, comprise reverse transcriptase PCR (RT-PCR) or, in further embodiments, array hybridization, wherein the array comprises an immobilized nucleic acid probe that specifically hybridizes TRPM1 mRNA, TRPM1 cDNA, or complements thereof. The expression level of TRPM1 may be assessed in further embodiments by evaluating the amount of TRPM1 protein in the melanoma sample.

In still another aspect of the present invention, a method is provided for treating a patient diagnosed as having melanoma comprising administering to the patient an effective amount of a therapeutic agent that increases miR-211 biological activity. In some embodiments, the miR-211 biological activity is increased in the melanoma cells. Such an increase may be, in some embodiments, by at least 10%, at least 50%, or at least 100%.

In further embodiments, the therapeutic agent may comprise a nucleic acid encoding miR-211. The nucleic acid may be encoded in a vector or a viral vector, or may be contained within a liposome. The administration of the therapeutic agent may additionally, in some embodiments, result in a reduction in the expression of KCNMA1.

In yet another aspect, the present invention provides a method for determining the proliferation rate of melanoma in a subject comprising: (i) assessing the expression level of miR-211 in a melanoma sample obtained from the subject; and (ii) identifying the proliferation rate of the melanoma, wherein a lower expression level of miR-211 in the sample indicates a greater proliferation rate and a higher expression level of miR-211 in the sample indicates a lower proliferation rate.

In some embodiments, the expression level of miR-211 is assessed by evaluating the amount of miR-211 mRNA in the melanoma sample. Such an evaluation may, in some embodiments, comprise reverse transcriptase PCR (RT-PCR) or, in further embodiments, array hybridization, wherein the array comprises an immobilized nucleic acid probe that specifically hybridizes miR-211 mRNA, miR-211 cDNA, or complements thereof.

In still another aspect of the present invention, a method is provided for determining the metastatic potential of melanoma in a subject comprising: (i) assessing the expression level of miR-211 in a melanoma sample obtained from the subject; and (ii) identifying the metastatic potential of the melanoma, wherein a lower expression level of miR-211 in the sample indicates a greater metastatic potential and a higher expression level of miR-211 in the sample indicates a lower metastatic potential.

In some embodiments, the expression level of miR-211 is assessed by evaluating the amount of miR-211 mRNA in the melanoma sample. Such an evaluation may, in some embodiments, comprise reverse transcriptase PCR (RT-PCR) or, in further embodiments, array hybridization, wherein the array comprises an immobilized nucleic acid probe that specifically hybridizes miR-211 mRNA, miR-211 cDNA, or complements thereof.

In another aspect, a method is provided for diagnosing a human as having melanoma or having an increased likelihood of melanoma, said method comprising, (i) determining, in a sample obtained from a human, the presence or absence of a TRPM1 gene promoter mutation that causes a reduction in the TRPM1 gene expression relative to the TRPM1 gene expression from a TRPM1 gene promoter lacking that mutation, and (ii) identifying the human has having melanoma or an increased likelihood of melanoma when a TRPM1 gene promoter mutation is identified, and identifying the human as not having melanoma or an increased likelihood of melanoma when the TRPM1 gene promoter mutation is absent. In some embodiments, the TRPM1 gene promoter mutation is selected from the group consisting of the T61C, C116T, A1430, A153G, 0331A, G708A, and T724C mutations relative to SEQ ID NO: 36. In further embodiments, the sample may comprise skin.

In yet another aspect, the present invention provides a method for treating a patient diagnosed as having melanoma comprising administering to the patient an effective amount of a therapeutic agent that increases TP53 biological activity. In some embodiments, the TP53 biological activity is increased in the melanoma cells. In further embodiments, the TP53 biological activity is increased at least 2-fold, at least 3-fold, or at least 5-fold. In still further embodiments, the therapeutic agent may further act to increase miR-211 expression. The therapeutic agent may increase the expression of TP53 mRNA or TP53 protein.

In still another aspect, a method is provided for treating a patient diagnosed as having melanoma comprising administering to the patient an effective amount of a therapeutic agent that reduces IGFBP5 biological activity. In some embodiments, the IGFBP5 biological activity is reduced in the melanoma cells. The biological activity may be reduced by at least 10%, at least 50%, or at least 90%. In further embodiments, the therapeutic agent may comprise an IGFBP5 siNA. The therapeutic agent may, in some embodiments, comprise a nucleic acid encoding miR-211. The nucleic acid may, in some embodiments, be encoded in a vector or a viral vector, or may be contained within a liposome. In still further embodiments, the therapeutic agent may reduce the expression of IGFBP5 mRNA or IGFBP5 protein, or, in some embodiments, may also increase MITF, TP53, or TRPM1 expression.

In still another aspect, a method is provided for diagnosing melanoma in a subject suspected of having melanoma comprising: (i) assessing the expression level of miR-211 in a biological sample obtained from the subject; (ii) comparing the expression level of miR-211 in the sample to the a reference expression level derived from the expression level of miR-211 in samples obtained from subjects diagnosed as not having melanoma; and (iii) identifying the subject as having melanoma when the expression level of miR-211 in the sample is less than the reference expression level or identifying the subject as not having melanoma when the expression level of miR-211 in the sample is not less than the reference expression level. In some embodiments, the biological sample may comprise skin, or, in further embodiments, skin epidermis or melanocytes. In still further embodiments, the expression level of miR-211 is assessed by evaluating the amount of miR-211 in RNA in the biological sample. In further embodiments, the miR-211 mRNA comprises reverse transcriptase PCR (RT-PCR). In still further embodiments, evaluation of the miR-211 mRNA may comprise hybridization, wherein the array comprises an immobilized nucleic acid probe that specifically hybridizes miR-211 mRNA, miR-211 cDNA, or complements thereof.

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof). Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “short interfering nucleic acid” or “siNA” refers to any nucleic acid molecule capable of down regulating (i.e., inhibiting) gene expression in a mammalian cells (preferably a human cell). siNA includes without limitation nucleic acid molecules that are capable of mediating sequence specific RNAi, for example short interfering RNA (siRNA), double-stranded RNA (dsRNA), micro-RNA (miRNA), and short hairpin RNA (shRNA).

As used herein, the term “KCNMA1 siRNA” refers to a short interfering nucleic acid as defined above that targets or preferentially binds to an mRNA encoded by KCNMA1.

As used herein, the term “increase in biological activity” refers to any measurable increase of any biological effect caused by an increase in the expression of a nucleic acid or protein. An increase in biological activity may often be measured by increased amounts of RNA (e.g., mRNA) or protein, or may be measured functionally.

As used herein, the term “diagnosing” means determining a disease state or condition in a patient (e.g., melanoma) in such a way as to inform a health care provider as to the necessity or suitability of a treatment for the patient.

As used herein, the term “miR-211” refers tee a small, non-coding nucleic acid molecule encoded in the sixth intron of the TRPM1 gene that targets mRNA encoded by KCNMA1. miR-211 may refer to any type of nucleic acid molecule including ribonucleotides, deoxyribonucleotides, or modified nucleotides.

As used herein, the term “sense region” refers to a nucleotide sequence of a siNA molecule complementary (partially or fully) to an antisense region of the siNA molecule. Optionally, the sense strand of a siNA molecule may also include additional nucleotides not complementary to the antisense region of the siNA molecule.

As used herein, the term “antisense region” refers to a nucleotide sequence of a siNA molecule complementary (partially or fully) to a target nucleic acid sequence. Optionally, the antisense strand of a siNA molecule may include additional nucleotides not complementary to the sense region of the siNA molecule.

As used herein, the term “duplex region” refers to the region in two complementary or substantially complementary oligonucleotides that form base pairs with one another that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and/or 3′ overhangs.

An “abasic nucleotide” conforms to the general requirements of a nucleotide in that it contains a ribose or deoxyribose sugar and a phosphate but, unlike a normal nucleotide, it lacks a base (i.e., lacks an adenine, guanine, thymine, cytosine, or uracil). Abasic deoxyribose moieties include, for example, abasic deoxyribose-3′-phosphate; 1,2-dideoxy-D-ribofuranose-3-phosphate; 1,4-anhydro-2-deoxy-D-ribitol-3-phosphate.

As used herein, the term “inhibit”, “down-regulate”, or “reduce,” with respect to gene expression, means that the level of RNA molecules encoding one or more proteins or protein subunits (e.g., mRNA) is reduced below that observed in the absence of the inhibitor. Expression may be reduced by at least 90%, 80%, 70%, 60%, 50%, 40%, 30%, 20%, 10%, 5% or below the expression level observed in the absence of the inhibitor.

### BRIEF DESCRIPTION OF THE FIGURES

FIG. 1 is a histogram showing log₂ of mean expression ratios of miRNA levels.

FIG. 2(A) is a series of bar graphs showing levels of individual miRNAs as measured by qRT-PCR in eight different melanoma cell lines, and (B) is a photograph of the northern blot analysis of miR-21 and miR-let-7g in five melanoma cell lines and melanocytes.

FIG. 3 is a bar graph showing normalized ratios of miR-211 levels in clinical samples relative to its level in a melanocyte cell line as measured by qRT-PCR.

FIG. 4 is a histogram of log₂ transformed expression ratios showing the fold change of mRNAs in WM1552C to those in a melanocyte cell line.

FIG. 5 is a series of bar graphs showing the effects of miR-211 overexpression on KCNMA1 gene expression.

FIG. 6(A) is a photograph of a Western blot analysis of KCNMA1 protein expression in melanocytes and melanoma cells; (B) is a bar graph showing the relative expression of KCNMA1 in mRNA in cells expressing and not expressing miR-211; (C) is a photograph of a Western blot analysis of KCNMA1 protein expression in WM1552C cell lines; (D) is a line graph of the inverse correlation between miR-211 and KCNMA1 protein levels; (E) is two photographs showing the inhibitory effect of miR-211 on KCNMA1 protein levels; and (F) is a bar graph showing the inhibitory effect of miR-211 on mRNA containing KCNMA1 3′-UTR sequences.

FIG. 7 is a bar graph showing the effect of MITF knock-down on TRPM1 and miR-211 expression in melanoma cells.

FIGS. 8(A) and (B) are line graphs of relative mean cell titers; (C) is a series of photographs of cell invasion assays; (D) is a bar graph showing the results of cell invasion assays; and (E) is a bar graph and photograph showing the effect on melanoma cell invasiveness by KCNMA1 protein expression.

FIG. 9 is an illustration showing a model summarizing the regulation and role of miR-211 in melanoma.

FIG. 10 is a depiction of the higher expression of miR-211 in melanocytes and nevus sample compared to normal skin.

FIG. 11 is a histogram showing miR-211 expression in stable melanoma cell lines compared to melanocytes.

FIG. 12 is a diagram showing mutagenesis of miR-211 target seed sequence in the 3′-UTR of KCNMA1. The wildtype 3′UTR is provided as SEQ ID NO: 25 and the mutant 3′UTR is provided as SEQ ID NO: 26.

FIG. 13 is a sequence alignment showing the upstream promoter of TRPM1 in melanocytes and the SKMEL-28, A375, and WM1152C melanoma cells lines. The promoter sequences are provided as SEQ ID NOs: 36-39, respectively. Point mutations in the TRPM1 promoters of the A375 and/or WM1152C cell lines are highlighted.

FIG. 14 is a bar graph showing the results of a luciferase reporter assay comparing two melanoma cell lines with both melanocyte and WM1552C TRPM1 reporters.

FIG. 15 is a series of bar graphs showing the results of a qPCR assay to determine expression of MITF (A), TRPM1 (B), IGFBP5 (C), and RUNX2 (D) after treatment with MITE siRNAs.

FIG. 16 is a chart showing Next Gen sequencing results of the IGFBP5 locus in WM1552C and WM1552C/211 cells.

FIG. 17 is a bar graph showing the results of a luciferase reporter assay conducted to determine the relative luminescence of a melanoma cell line, vector only, or miR-211 expressing cells transfected with either native or mutant 3′UTR reporter constructs.

FIG. 18 is a series of bar graphs showing the expression of MITE (A), TRPM1 (B), and IGFBP5 (C) after treatment with TP53 siRNAs.

FIG. 19 is a bar graph showing the results of a cell count of A375 and A375/211 cells after treatment with 0 nM DFO, 250 nM DFO, or 400 nM DFO.

FIG. 20 is a bar graph showing the results of a cell count of A375 and A375/211 cells in hypoxic and normoxic conditions.

FIG. 21 is a bar graph showing the results of a cell count of WM1552C and WM1552C/211 cells after treatment with 0 nM DFO, 250 nM DFO, or 400 nM DFO.

FIG. 22 is a bar graph showing the results of a cell count of WM1552C and WM1552C/211 cells in hypoxic and normoxic conditions.

FIG. 23 is a bar graph showing the results of a cell count of SKEML-28 and SKEML-28/211 cells after treatment with 0 nM DFO, 250 nM DFO, or 400 nM DFO.

FIG. 24 is a bar graph showing the results of a qRT-PCR assay of melanocytes and SKMEL-28 cells under hypoxic or simulated hypoxic conditions.

FIG. 25 is a bar graph comparing the PI species (acyl chains composition) of A375 cells to A375/211 cells.

FIG. 26 is the nucleic acid sequence of the 3′-UTR of the IGFBP5 cDNA.

### DETAILED DESCRIPTION

The immediate molecular mechanisms behind invasive melanoma are poorly understood. Recent studies implicate microRNAs (miRNAs) as important agents in melanoma and other cancers. To investigate the role of miRNAs in melanoma, human melanoma cell lines were subjected to miRNA expression profiling, and a range of variations in several miRNAs was reported. Specifically, compared with expression levels in melanocytes, levels of miR-211 were consistently reduced in all eight non-pigmented melanoma cell lines we examined; they were also reduced in 21 out of 30 distinct melanoma samples from patients, classified as primary in situ, regional metastatic, distant metastatic, and nodal metastatic. The levels of several predicted target mRNAs of miR-211 were reduced in melanoma cell lines that ectopically expressed miR-211.

In vivo target cleavage assays confirmed one such target mRNA encoded by KCNMA1. Mutating the miR-211 binding site seed sequences at the KCNMA1 3′-UTR abolished target cleavage. KCNMA1 mRNA and protein expression levels varied inversely with miR-211 levels. Two different melanoma cell lines ectopically expressing miR-211 exhibited significant growth inhibition and reduced invasiveness compared with the respective parental melanoma cell lines. An shRNA against KCNMA1 mRNA also demonstrated similar effects on melanoma cells.

miR-211 is encoded within the sixth intron of TRPM1, a candidate suppressor of melanoma metastasis. The transcription factor MITF, important for melanocyte development and function, is needed for high TRPM1 expression. MITF is also needed for miR-211 expression, suggesting that the tumor-suppressor activities of MITF and/or TRPM1 may at least partially be due to miR-211's negative post transcriptional effects on the KCNMA1 transcript. Given previous reports of high KCNMA1 levels in metastasizing melanoma, prostate cancer and glioma, our findings that miR-211 is a direct posttranscriptional regulator of KCNMA1 expression as well as the dependence of this miRNA's expression on MITF activity, establishes miR-211 as an important regulatory agent in human melanoma.

The reduced expression of miR-211 in these cell lines can be seen in clinical isolates of human melanomas. Further, there is evidence that a principal effect of the reduced expression of miR-211 is the increased expression of its target transcript KCNMA1. The expression of KCNMA1, encoding a calcium ion-regulated potassium channel protein, appears to at least partially account for the high cell proliferation rate and invasiveness of melanoma cell lines. MITF expression is also important for the coordinate expression of miR-211, and TRPM1. TRPM1 gene is a suppressor of melanoma metastasis, which encodes a transient receptor potential family member calcium channel protein, and encodes miR-211 in its sixth intron.

Current understanding of the molecular mechanisms of carcinogenesis is beginning to include not only the role of protein coding genes but also that of non-coding regulatory RNA, especially miRNAs. In the case of melanoma, the discovery of miRNAs whose expression levels are reduced in melanoma cells can lead to the identification of genes that are responsible for oncogenesis and invasiveness. Along that line, it is shown herein that miR-211 levels are consistently reduced in melanoma cells compared to its levels in melanocytes, and that the expression levels of several potential miR-211 target mRNAs are elevated in melanoma cells. The increased expression of one confirmed target transcript in particular, KCNMA1, is associated with high invasiveness and proliferation in melanoma cells in vitro.

It is likely that the down-regulation of miR-211 causes elevated levels of KCNMA1 protein in melanoma cells, which at least in part explains the invasiveness of malignant melanoma. Additionally, melanoma cell lines engineered to express high levels of miR-211 begin to lose expression shortly after removal from selection, indicating a strong bias against miR-211 expression during the growth of melanoma cell lines and suggests that the rapid proliferation of melanoma cells in culture is directly related to low miR-211 activity in these cells. However, without wishing to be hound by any theory, it is possible that as yet unidentified targets of miR-211 (besides KCNMA1) may have a positive feedback effect on KCNMA1 levels and are responsible for invasiveness. An alternative possibility is that miR-211 down-regulation in melanoma causes other transformational events unrelated to KCNMA1, leading to higher oncogenesis and invasiveness. Both of these more complex possibilities are consistent with some evidence, but not with the full set of data presented herein.

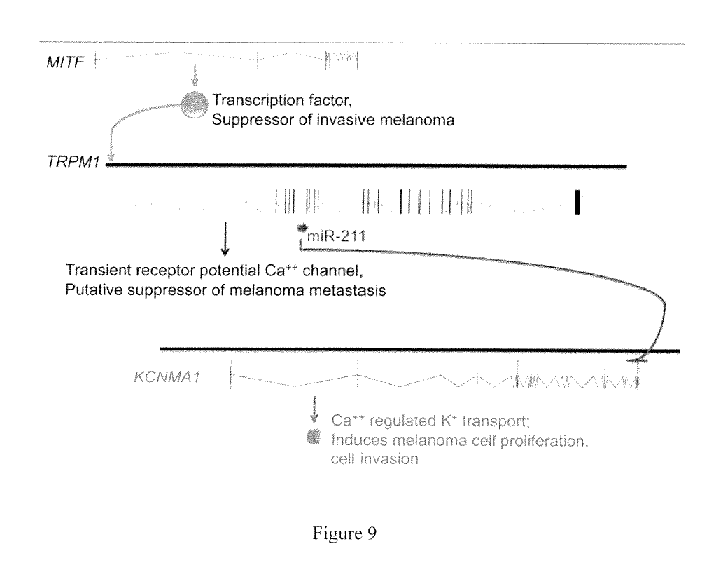

The transcription factor MITF, which regulates the expression of TRPM1, is also needed for high-level expression of miR-211. Thus, the regulation by MITF of both TRPM1 and miR-211 genes can be speculated to have similar effects on melanoma invasiveness separately through their respective gene products: the former a Ca⁺⁺ channel protein (TRPM1), and the latter a miRNA targeted against the Ca⁺⁺ regulated K⁺ channel protein KCNMA1. Thus, the invasiveness of melanoma cells could partly be the result of the breakdown of processes related to calcium-regulated ion homeostasis. The recent finding that salinomycin, an inhibitor of transport, is a selective inhibitor of cancer stem cell proliferation is consistent with our findings on the role of KCNMA1 in melanoma cells [63]. We cannot eliminate the formal possibility that the potential tumor suppressor activity of TRPM1 gene is, at least in part, due to the co-expression of miR-211 encoded from within its sixth intron. FIG. 9 provides a summary of a simple model of the putative mechanism of development of invasive melanoma, which highlights the role of miR-211.

In contrast to the downregulation of miR-211 levels in most melanoma cells and clinical samples shown herein, Gaur et al. [64] previously reported that miR-211 was over-expressed in 6 of 8 tested melanoma lines from the NCI-60 panel of cancer cells. However, a leave-one-out sensitivity analysis conducted by Gaur et al. [64] failed to show a significant effect on the confidence interval when miR-211 expression level was omitted, suggesting low specificity or sensitivity with respect to miR-211 in those experiments. Muller et al. [41] compared miRNA expression in melanoma cell lines with pooled normal human epidermal melanocytes; miR-211 was not down-regulated in their study. It is likely that the melanocyte cells (pooled epidermal melanocytes) used in the latter studies were physiologically and genetically different from the melanocyte lines used herein. Jukic et al., [44] reported that miR-211 was up-regulated in nevi and dramatically down-regulated in metastatic melanoma compared to nevi controls. These results correspond with the results shown herein and contradict the results published by Schultz, et al., [31].

Given that miR-211 is down-regulated in non-pigmented melanoma and its expression is regulated by the MITF gene, the down-regulation of miR-211 and the corresponding up-regulation of its target transcript KCNMA1 are therefore important molecular events for melanoma development and/or progression.

RNA Interference and siNA

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). Post-transcriptional gene silencing is believed to be an evolutionarily-conserved cellular mechanism for preventing expression of foreign genes that may be introduced into the host cell (Fire et al., 1999, Trends Genet., 15, 358). Post-transcriptional gene silencing may be an evolutionary response to the production of double-stranded RNAs (dsRNAs) resulting from viral infection or from the random integration of transposable elements (transposons) into a host genome. The presence of dsRNA in cells triggers the RNAi response that appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of dicer, a ribonuclease III enzyme (Bass, 2000, Cell, 101, 235; Zamore et al., 2000, Cell, 101, 25-33; Hammond et al., 2000, Nature, 404, 293). Dicer processes long dsRNA into double-stranded short interfering RNAs (siRNAs) which are typically about 21 to about 23 nucleotides in length and include about 19 base pair duplexes (Zamore et al., 2000, Cell, 101, 25-33; Bass, 2000, Cell. 101, 235; Elbashir et al, 2001, Genes Dec. 15, 188).

Single-stranded RNA, including the sense strand of siRNA, trigger an RNAi response mediated by an endonuclease complex known as an RNA-induced silencing complex (RISC). RISC mediates cleavage of this single-stranded RNA in the middle of the siRNA duplex region (i.e., the region complementary to the antisense strand of the siRNA duplex) (Elbashir et al., 2001, Genes Dev., 15, 188).

In certain embodiments, the siNAs may be a substrate for the cytoplasmic Dicer enzyme a “Dicer substrate”) which is characterized as a double stranded nucleic acid capable of being processed in vivo by Dicer to produce an active nucleic acid molecules. The activity of Dicer and requirements for Dicer substrates are described, for example, U.S. 2005/0244858. Briefly, it has been found that dsRNA, having about 25 to about 30 nucleotides, effective result in a down-regulation of gene expression. Without wishing to be bound by any theory, it is believed that Dicer cleaves the longer double stranded nucleic acid into shorter segments and facilitates the incorporation of the single-stranded cleavage products into the RNA-induced silencing complex (RISC complex). The active RISC complex, containing a single-stranded nucleic acid cleaves the cytoplasmic RNA having complementary sequences.

It is believed that Dicer substrates must conform to certain general requirements in order to be processed by Dicer. The Dicer substrates must of a sufficient length (about 25 to about 30 nucleotides) to produce an active nucleic acid molecule and may further include one or more of the following properties: (i) the dsRNA is asymmetric, e.g. has a 3′ overhang on the first strand (antisense strand) and (ii) the dsRNA has a modified 3′ end on the antisense strand (sense strand) to direct orientation of Dicer binding and processing of the dsRNA to an active siRNA. The Dicer substrates may be symmetric or asymmetric. For example, Dicer substrates may have a sense strand includes 22-28 nucleotides and the antisense strand may include 24-30 nucleotides, resulting in duplex regions of about 25 to about 30 nucleotides, optionally having 3′-overhangs of 1-3 nucleotides.

Dicer substrates may have any modifications to the nucleotide base, sugar or phosphate backbone as known in the art and/or as described herein for other nucleic acid molecules (such as siNA molecules).

The RNAi pathway may be induced in mammalian and other cells by the introduction of synthetic siRNAs that are 21 nucleotides in length (Elbashir et al., 2001, Nature, 411, 494 and Tuschl et al., WO 01/75164; incorporated by reference in their entirety). Other examples of the requirements necessary to induce the down-regulation of gene expression by RNAi are described in Zamore et al., 2000, Cell, 101, 25-33; Bass, 2001, Nature, 411, 428-429; Kreutzer et al., WO 00/44895; Zernicka-Goetz et al., WO 01/36646; Fire, WO 99/32619; Plaetinck et al., WO 00/01846; Mello and Fire, WO 01/29058; Deschamps-Depaillette, WO 99/07409; and Li et al., WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831; each of which is hereby incorporated by reference in its entirety.

Briefly, an siNA nucleic acid molecule can be assembled from two separate polynucleotide strands (a sense strand and an antisense strand) that are at least partially complementary and capable of forming stable duplexes. The length of the duplex region may vary from about 15 to about 49 nucleotides (e.g., about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides). Typically, the antisense strand includes nucleotide sequence that is complementary to nucleotide sequence in a target nucleic acid molecule. The sense strand includes nucleotide sequence corresponding to the target nucleic acid sequence which is therefore at least substantially complementary to the antisense stand. Optionally, an siNA is “RISC length” and/or may be a substrate for the Dicer enzyme. Optionally, an siNA nucleic acid molecule may be assembled from a single polynucleotide, where the sense and antisense regions of the nucleic acid molecules are linked such that the antisense region and sense region fold to form a duplex region (i.e., forming a hairpin structure).

### 5′ Ends, 3′ Ends and Overhangs

siNAs may be blunt-ended on both sides, have overhangs on both sides or a combination of blunt and overhang ends. Overhangs may occur on either the 5′- or 3′-end of the sense or antisense strand. Overhangs typically consist of 1-8 nucleotides (e.g., 1, 2, 3, 4, 5, 6, 7, or 8 nucleotides each) and are not necessarily the same length on the 5′- and 3′-end of the siNA duplex. The nucleotide(s) forming the overhang need not be of the same character as those of the duplex region and may include deoxyribonucleotide(s), ribonucleotide(s), natural and non-natural nucleobases or any nucleotide modified in the sugar, base or phosphate group such as disclosed herein.

The 5′- and/or 3′-end of one or both strands of the nucleic acid may include a free hydroxyl group or may contain a chemical modification to improve stability. Examples of end modifications (e.g., terminal caps) include, but are not limited to, abasic, deoxy abasic, inverted (deoxy) abasic, glyceryl, dinucleotide, acyclic nucleotide, amino, fluoro, chloro, bromo, CN, CF, methoxy, imidazole, carboxylate, thioate, C1 to C10 lower alkyl, substituted lower alkyl, alkaryl or aralkyl, OCF3, OCN, O-, S-, or N-alkyl; O-, S-, or N-alkenyl; SOCH3; SO2CH3; ONO2; NO2, N3; heterocycloalkyl; heterocycloalkaryl; aminoalkylamino; polyalkylamino or substituted silyl, as, among others, described in European patents EP 586,520 and EP 618,925.

### Chemical Modifications

siNA molecules optionally may contain one or more chemical modifications to one or more nucleotides. There is no requirement that chemical modifications are of the same type or in the same location on each of the siNA strands. Thus, each of the sense and antisense strands of an siNA may contain a mixture of modified and unmodified nucleotides. Modifications may be made for any suitable purpose including, for example, to increase RNAi activity, increase the in vivo stability of the molecules (e.g., when present in the blood), and/or to increase bioavailability.

Suitable modifications include, for example, internucleotide or internucleoside linkages, dideoxyribonucleotides, 2′-sugar modification including amino, fluoro, methoxy, alkoxy and alkyl modifications; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC). 2′,3′-didehydro-2′,3′-dideoxythymidi-ne (d4T) and the monophosphate nucleotides of 3% azido-3′-deoxythymidine (AZT), 2′,3′-dideoxy-3′-thiacytidine (3TC) and 2′-didehydro-2′,3′-dide-oxythymidine (d4T).

Other suitable modifications include, for example, locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides (WO 00/47599, WO 99/14226, WO 98/39352, and WO 2004/083430).

Chemical modifications also include terminal modifications on the 5′ and/or 3′ part of the oligonucleotides and are also known as capping moieties. Such terminal modifications are selected from a nucleotide, a modified nucleotide, a lipid, a peptide, and a sugar.

Chemical modifications also include L-nucleotides. Optionally, the L-nucleotides may further include at least one sugar or base modification and/or a backbone modification as described herein.

### Delivery of Nucleic Acid-Containing Pharmaceutical Formulations

Nucleic acid molecules disclosed herein (including siNAs and Dicer substrates) may be administered with a carrier or diluent or with a delivery vehicle which facilitate entry to the cell. Suitable delivery vehicles include, for example, viral vectors, viral particles, liposome formulations, and lipofectin.

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995), Maurer et al., Mol. Membr. Biol., 16: 129-140 (1999); Hofland and Huang, Handb, Exp. Pharmacol., 137: 165-192 (1999): and Lee et al., ACS Symp. Ser., 752: 184-192 (2000); U.S. Pat. Nos. 6,395,713; 6,235,310; 5,225,182; 5,169,383; 5,167,616; 4,959217; 4,925,678; 4,487,603; and 4,486,194; WO 94/02595; WO 00/03683; WO 02/08754; and U.S. 2003/077829.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); WO 03/47518; and WO 03/46185), polylactic-co-glycolic)acid (PLGA) and PLCA microspheres (see for example U.S. Pat. No. 6,447,796 and U.S. 2002/130430), biodegradable nanocapsules, and bioadhesive microspheres, or by proteinaceous vectors (WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5: 2330-2337 (1999) and WO 99/31262. The molecules of the instant invention can be used as pharmaceutical agents.

Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly (ethylene glycol) lipids (PEG-modified, or long-circulating liposomes or stealth liposomes).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see, for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; U.S. Pat. No. 6,586,524 and U.S. 2003/0077829).

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Therapeutic nucleic acid molecules may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors are capable of expressing the nucleic acid molecules either permanently or transiently in target cells. Delivery of nucleic acid molecule expressing vectors can be systemic, such as by intravenous, subcutaneous, or intramuscular administration.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine. An expression vector may encode one or both strands of a nucleic acid duplex, or a single self-complementary strand that self hybridizes into a nucleic acid duplex. The nucleic acid sequences encoding nucleic acid molecules can be operably linked to a transcriptional regulatory element that results expression of the nucleic acid molecule in the target cell, Transcriptional regulatory elements may include one or more transcription initiation regions (e.g., eukaryotic pol I, II or III initiation region) and/or transcription termination regions (e.g., eukaryotic pol I, II or III termination region). The vector can optionally include an open reading frame (ORF) for a protein operably linked on the 5′ side or the 3′-side of the sequence encoding the nucleic acid molecule; and/or an intron (intervening sequences).

The nucleic acid molecules or the vector construct can be introduced into the cell using suitable formulations. One preferable formulation is with a lipid formulation such as in Lipofectamine™ 2000 (Invitrogen, CA, USA), vitamin A coupled liposomes (Sato et al. Nat Biotechnol 2008; 26:431-142, PCT Patent Publication No. WO 2006/068232). Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA in a buffer or saline solution and directly inject the formulated dsRNA into cells, as in studies with oocytes. The direct injection of dsRNA duplexes may also be done. Suitable methods of introducing dsRNA are provided, for example, in U.S. 2004/0203145 and U.S. 20070265220.

Polymeric nanocapsules or microcapsules facilitate transport and release of the encapsulated or bound dsRNA into the cell. They include polymeric and monomeric materials, especially including polybutylcyanoacrylate. The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid moles may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system. Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

### EXAMPLES

The present methods, thus generally described, will be understood more readily by reference to the following examples, which are provided by way of illustration and are not intended to be limiting of the present methods and kits.

### Example 1

### miR-211 is Expressed at a Low Level in Non-Pigmented Melanoma Cell Lines

The human epidermal melanocyte cell line HEM-1 (ScienCell™, Catalog #2200) and primary epidermal melanocytes—neonatal (ATCC—PCS-200-012) were grown in MelM media containing MelGS growth supplements, 0.5% FBS, and pen/strep solution. The melanoma cell lines examined included: A375 (stage 4, ATCC® Number: CRL-1619), G361 (stage 4, ATCC), LOX-IMV1 (stage 4, ATCC), HT-144 (stage 4, ATCC® Number: HTB-63), RPMI-7951 (stage 4, ATCC® Number: HTB-66), SK-MEL2 (stage 4, ATCC), SK-MEL28 (stage 3, ATCC), WM793B (stage 1, ATCC® Number: CRL-2806), and WM1552C (stage 3, ATCC® Number: CRL-2808). All melanoma cell lines were grown in Complete Tu Media containing a 4:1 mixture of MCDB-153 medium with 1.5 g/L sodium bicarbonate and Leibovitz's L-15 medium with 2 mM L-glutamine, 2% FBS, and 1.68 mM CaCl₂. Information regarding all clinical samples, derived from frozen samples, is described in Table 1.

TABLE 1

| Clinical Sample # | Tumor Type |
|---|---|
| 1 | Nodal Metastasis |
| 2 | Nodal Metastasis |
| 3 | Regional Metastasis |
| 4 | Nodal Metastasis |
| 5 | Nodal Metastasis |
| 6 | Regional Metastasis |
| 7 | Nodal Metastasis |
| 8 | Nodal Metastasis |
| 9 | Distant Metastasis |
| 10 | Primary Melanoma |
| 11 | Nodal Metastasis |
| 12 | Nodal Metastasis |
| 13 | Distant Metastasis |
| 14 | Primary Melanoma |
| 15 | Nodal Metastasis |
| 16 | Nodal Metastasis |
| 17 | Distant Metastasis |
| 18 | Distant Metastasis |
| 19 | Nodal Metastasis |
| 20 | Nodal Metastasis |
| 21 | Primary Melanoma |
| 22 | Primary Melanoma |
| 23 | Primary Melanoma |
| 24 | Primary Melanoma |
| 25 | Distant Metastasis |
| 26 | Distant Metastasis |
| 27 | Regional Metastasis |
| 28 | Regional Metastasis |
| 29 | Regional Metastasis |
| 30 | Regional Metastasis |

miRNA NCode™ version 2 array (Invitrogen) containing 553 human and 427 mouse miRNAs, and the TILDA array (ABI) were used for miRNA expression profiling. The miRNA samples were labelled with AlexaFluor® conjugated dendrimers using the direct labelling kit (Genisphere). Hybridization conditions were routinely assessed by discriminating between 2 nt variants at internal sites, and most probes can distinguish between 1 nt variants. The arrays were scanned with Axon B-4000 (Agilent).

Expression levels of all statistically significant and differentially expressed mRNAs and miRNAs were confirmed by qRT-PCR using TaqMan® expression kits (Applied Biosystems) [65] using multiple technical and biological replicates. GAPDH was used as the internal reference probe for normalization of expression values of mRNA, and RNU48 was used for normalization of miRNA. RNA analysis by Northern blots used 20 μg of total RNA concentrated from each sample (melanoma cell lines and melanocytes), separated on 15% urea denaturing polyacrylamide gels by electrophoresis. Gels were electroblotted to nylon membranes, cross-linked by UV, prehybridized in ULTRAhyb®-Oligo (Ambion) for 30 minutes at 42° C., and hybridized with 5′-biotinylated anti-miRNA DNA oligonucleotides (100 nM each) at 42° C. overnight, washed, and detected by chemiluminescence (Brightstar® detection kit, Ambion). Anti-U6 probes were used as a reference control (at 10 pM).

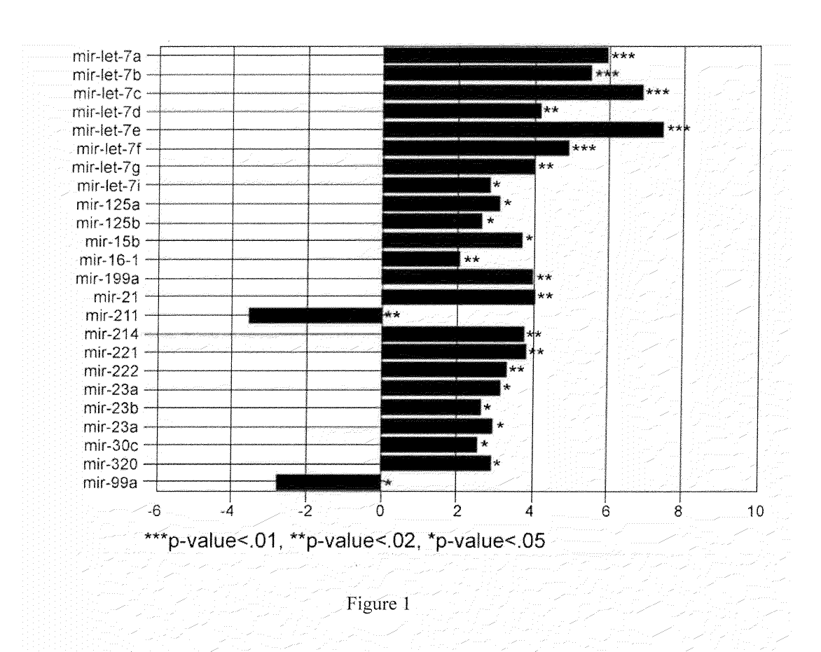

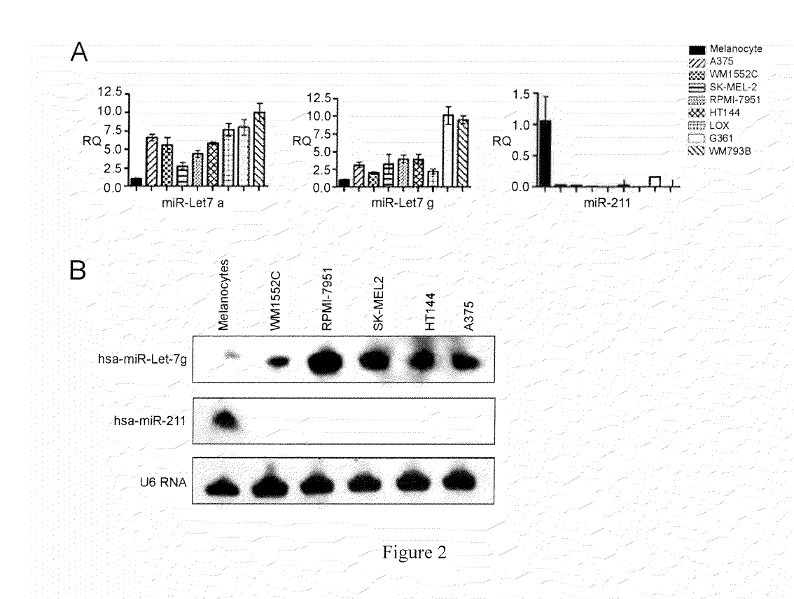

As the first step in identifying down-regulated miRNAs in human melanoma, significantly differentially expressed miRNA species were identified in the melanoma cell WM1552C (originally established from a stage 3 skin melanoma of a 72-year-old patient) compared to those in the normal melanocyte cell line HEM-1 by hybridization of total RNA samples to miRNA probe arrays (see Methods). FIG. 1 lists 24 statistically significant differentially expressed miRNAs, classified into three groups according to their significance levels (P <0.01, 0.02, and 0.05, respectively) (see also Table 2). To address whether the differential miRNA expression levels observed with WM1552C varied among other established melanoma cell lines, we performed quantitative reverse transcriptase mediated polymerase chain reaction (qRT-PCR) analysis on RNA isolated from WM1552C and seven additional non-pigmented melanoma cell lines (FIG. 2A), addressing the expression levels of three separate microRNAs: miR-let7a, miR-let7g, which were over-expressed, and miR-211 was down-regulated. Northern blot analysis further confirmed these results (FIG. 2B). This consistency provided the opportunity to address the significance of the reduced level of miR-211 in melanoma. Next, the role of their target genes that are thus up-regulated in melanoma was determined. miR-211 showed the most robust and consistent changes in expression levels between melanocytes and non-pigmented melanoma cell lines. Results reported in FIGS. 1-2 implicate several additional miRNAs in melanoma that will not be discussed herein.

TABLE 2

| | | |
|---|---|---|
| SEQ ID NO: 1 | miR-let-7a | UGAGGUAGUAGGUUGUAUAGUU |
| SEQ ID NO: 2 | miR-let-7b | UGAGGUAGUAGGUUGUGUGGUU |
| SEQ ID NO: 3 | miR-let-7c | UGAGGUAGUAGGUUGUAUGGUU |
| SEQ ID NO: 4 | miR-let 7d | AGAGGUAGUAGGUUGCAUAGUU |
| SEQ ID NO: 5 | miR-let-7e | UGAGGUAGGAGGUUGUAUAGUU |
| SEQ ID NO: 6 | miR-let-7f | UGAGGUAGUAGAUUGUAUAGUU |
| SEQ ID NO: 7 | miR-let-7g | UGAGGUAGUAGUUUGUACAGUU |
| SEQ ID NO: 8 | miR-let-7i | UGAGGUAGUAGUUUGUGCUGUU |
| SEQ ID NO: 9 | miR-125a | UCCCUGAGACCCUUUAACCUGUGA |
| SEQ ID NO: 10 | miR-125b | UCCCUGAGACCCUAACUUGUGA |
| SEQ ID NO: 11 | miR-15b | UAGCAGCACAUCAUGGUUUACA |
| SEQ ID NO: 12 | miR-16-1 | UAGCAGCACGUAAAUAUUGGCG |
| SEQ ID NO: 13 | miR-199a | CCCAGUGUUCAGACUACCUGUUC |
| SEQ ID NO: 14 | miR-21 | UAGCUUAUCAGACUGAUGUUGA |
| SEQ ID NO: 15 | miR-211 | UUCCCUUUGUCAUCCUUCGCCU |
| SEQ ID NO: 16 | miR-214 | ACAGCAGGCACAGACAGGCAGU |
| SEQ ID NO: 17 | miR-221 | AGCUACAUUGUCUGCUGGGUUUC |
| SEQ ID NO: 18 | miR-222 | AGCUACAUCUGGCUACUGGGU |
| SEQ ID NO: 19 | miR-23a | AUCACAUUGCCAGGGAUUUCC |
| SEQ ID NO: 20 | miR-23b | AUCACAUUGCCAGGGAUUACC |
| SEQ ID NO: 21 | miR-26a | CCUAUUCUUGGUUACUUGCACG |
| SEQ ID NO: 22 | miR-30c | UGUAAACAUCCUACACUCUCAGC |
| SEQ ID NO: 23 | miR-320 | AAAAGCUGGGUUGAGAGGGCGA |
| SEQ ID NO: 24 | miR-99a | AACCCGUAGAUCCGAUCUUGUG |

### Example 2

### miR-211 Levels in Clinical Melanoma Samples

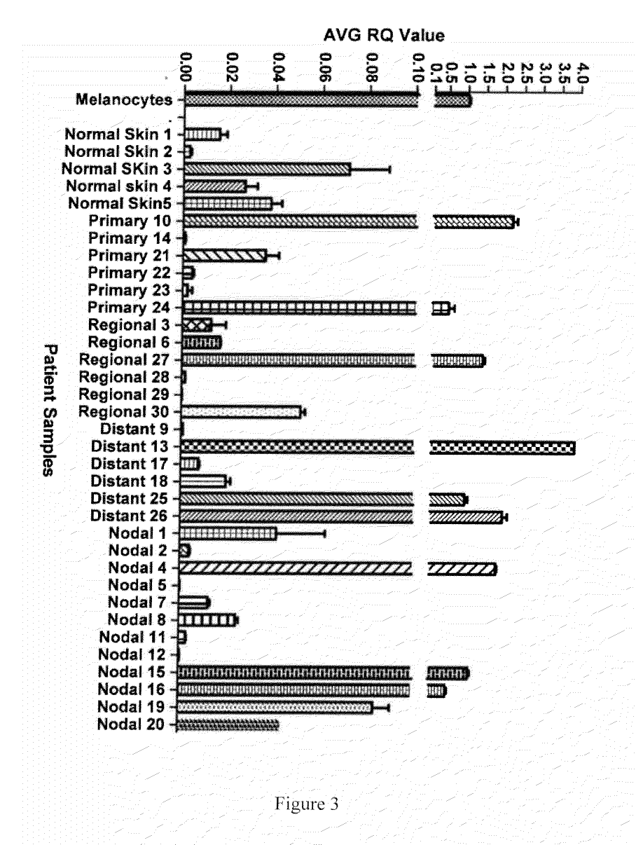

miR-211 transcript levels were assayed by qRT-PCR in 30 clinical melanoma samples (six primary, six regional, 12 nodal and six distal metastatic, respectively; described in Table S1). miR-211 expression levels were reduced in 21 of these clinical samples compared to that observed in melanocytes (FIG. 3, Table 3). In the remaining nine melanomas, six (one primary, one regional, two distant, and two nodal metastatic melanomas) showed statistically significant increases in miR-211 expression, whereas expression was not significantly different in the remaining samples. These samples were obtained from different patients; therefore, the observed differences may reflect different processes in melanoma development and progression, individual genetic differences, different proportions of non-melanoma (including non-pigmented) cells in the tumor samples, or a combination of these factors. miR-211 levels were low in the majority (21/30) of the tested melanoma clinical samples, a statistically significant trend (P=0.029, for random distribution by Fisher's exact test) that is consistent with the uniformly low expression levels in all eight melanoma-derived cell lines we studied. Note that miR-211 expression levels were also observed to be low in normal skin samples, which is expected given that melanocytes constitute a minor fraction of skin cells. Additional miRNAs that were over-expressed in melanoma cell lines relative to those in melanocytes were also over-expressed in the clinical melanoma samples but not in the normal skin samples (data not shown), confirming that normal skin samples are not the ideal background controls.

TABLE 3
miR-211

| Sample | Avg RQ | RQ St Dev |
|---|---|---|
| Melanocyte | 1.00063 | 0.04371 |
| Primary Melanoma 10 | 2.2066′S | 0.12293 |
| Primary Melanoma 14 | 0.00093 | 0.00041 |
| Primary Melanoma 21 | 0.03565 | 0.00577 |
| Primary Melanoma 22 | 0.00419 | 0.00086 |
| Primary Melanoma 23 | 0.00226 | 0.00189 |
| Primary Melanoma 24 | 0.52232 | 0.15612 |
| Mean Primary Melanoma | 0.02669 | 0.00647 |
| Regional Metastasis 3 | 0.01264 | 0.00628 |
| Regional Metastasis 6 | 0.0164 | 0.00033 |
| Regional Metastasis 27 | 1.44156 | 0.06496 |
| Regional Metastasis 28 | 0.00155 | 0.00016 |
| Regional Metastasis 29 | 0.00021 | 0.00006 |
| Regional Metastasis 30 | 0.05138 | 0.00201 |
| Mean Regional Metastases | 0.01309 | 0.00116 |
| Distant Metastasis 9 | 0.00095 | 0.00011 |
| Distant Metastasis 13 | 3.93166 | 0.07708 |
| Distant Metastasis 17 | 0.00774 | 0.00049 |
| Distant Metastasis 18 | 0.01958 | 0.00208 |
| Distant Metastasis 25 | 1.00047 | 0.09279 |
| Distant Metastasis 26 | 2.02149 | 0.13435 |
| Mean Distant Metastases | 0.10219 | 0.00692 |
| Nodal Metastasis 1 | 0.04167 | 0.02092 |
| Nodal Metastasis 2 | 0.0042 | 0.00047 |
| Nodal Metastasis 4 | 1.86626 | 0.0325 |
| Nodal Metastasis 5 | 0.00037 | 0.00008 |
| Nodal Metastasis 7 | 0.01243 | 0.00095 |
| Nodal Metastasis 8 | 0.02438 | 0.00144 |
| Nodal Metastasis 11 | 0.00318 | 0.00034 |
| Nodal Metastasis 12 | 0.00061 | 0.00012 |
| Nodal Metastasis 15 | 1.15988 | 0.04215 |
| Nodal Metastasis 16 | 0.55816 | 0.0367 |
| Nodal Metastasis 19 | 0.08403 | 0.00731 |
| Nodal Metastasis 20 | 0.04442 | 0.00172 |
| Mean Nodal Metastases | 0.02729 | 0.00233 |
| Normal Skin 1 | 0.01538 | 0.0033 |
| Normal Skin 2 | 0.00272 | 0.00067 |
| Normal Skin 3 | 0.07133 | 0.01717 |
| Normal Skin 4 | 0.02671 | 0.00515 |
| Normal Skin 5 | 0.03778 | 0.00469 |
| Mean Normal Skin | 0.01976 | 0.00391 |

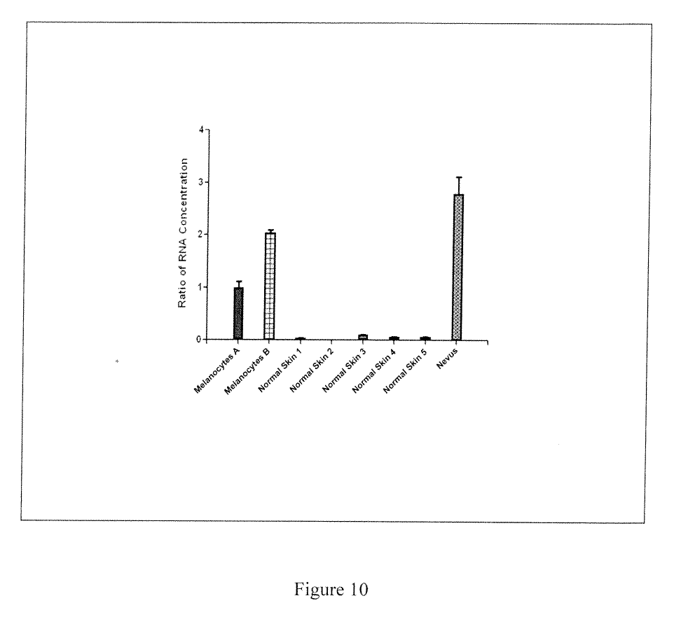

Although there is no perfect “normal” counterpart tissue for melanoma in clinical skin samples, miR-211 expression levels in additional melanocyte cell lines and in five independent isolates of normal skin samples were tested for comparison. Results show that miR-211 is elevated in both melanocyte cell lines compared to normal human skin (FIG. 10). miR-211 expression levels in pooled samples of nevi also agree with previously published results, supporting the observation that miR-211 is highly expressed in nevi compared to melanoma [44]. These observations are consistent with the understanding that nevi are composed of melanocytes. Together, these results suggest that the development of most melanomas is specifically associated with the depletion of miR-211 transcript levels. An alternative formal interpretation, which is unlikely considering the absence of supporting literature, is that the low miR-211 level in melanoma reflects a cellular origin of melanoma which is distinct from that of melanocytes.

### Example 3

### Stable Ectopic Expression of miR-211 in Melanoma Cell Lines Depletes Select Target Transcripts

For the initial transformation of miRNA array data, the GenePixPro 6.0 global normalization method was employed in which images and results are normalized together. Statistical significance tests were Welsh t-test, nonparametric ANOVA, (e.g., to select genes that have significantly less within sample variance compared to between sample variance), and correlation analysis with Pearson's product moment r and Spearman's r. Analysis was controlled for false discovery rate using q-values, with a priori cut off point of 10 percent [66, 67]. For mRNA expression array data, commencing with GeneChip® Human Exon 1.0 ST Array (Affymetrix, Inc.) four probes per exon and roughly 40 probes per gene, 7 total arrays were analysed (three arrays for melanocyte RNA, and four arrays for melanoma RNA). Cell files were loaded into Partek® Genomics Suite™ (Partek, Inc. St. Louis, Mo., USA) under the following algorithm constraints: interrogating probes selection, RMA background correction, adjusted for GC content, quintile normalization, log probes using base 2, with probe set summarization of median polish. Quality control assessment indicated clear separation based on the cell type. Gene level analysis use an ANOVA model; yⱼ=μ+Tⱼ+ε, where μ is the mean expression of the gene, Tⱼ is the tissue type, and ε is the error term. The ANOVA model generated a significance level for each probe set, along with the fold change, and imputed gene annotations. miR-211 target set of genes were obtained from public databases [miRanda, miRbase, miRNAmap, Tarbase, PicTar, Target ScanS, and DIANA MicroTest] and the results from ANOVA were matched to obtain the final target gene list of genes. This target list was imported into Ingenuity Pathway Analysis Version 6.0-1202 (Ingenuity Systems®). A core analysis was run employing direct relationships only, the Ingenuity knowledge base genes as the reference set, and with down-regulators as the defined expression value parameter. All microarray data have been deposited into GEO, and accession number is pending.

Oligonucleotides complementary to the miR-211 genomic sequences (miR-211 pre For—ttccctttgtcatccttcgcct (SEQ ID NO.: 27) and miR-211 pre Rev—aggcgaaggatgacaaagggaa (SEQ ID NO.: 28), containing HindIII and BamHI sites on their respective 5′ and 3′ ends) were used to amplify the 110 bp pre-m/R-211 sequence from human melanocyte genomic DNA (Amplitaq Gold®, Applied Biosystems) and TOPO®-cloned into the pCR®4-TOPO® vector (Invitrogen). The construct was sequenced, and the pre-hsa-miR-211 fragment was sub-cloned into pcDNA4/myc-HisA (Invitrogen) to create pcDNA4/miR-211. The KCNMA1 siRNA sequence was derived from Silencer® siRNA (Ambion, siRNA ID: 112882) and constructed as long complementary oligos (KCNMA1si For—cgtacttcaatgacaatatttcaagagaatattgtcattgaagtacgtctttttt (SEQ ID NO.: 29) and KCNMA1 si Rev—aaaaaagacgtacttcaatgacaatattctcttgaaatattgtcattgaagtacg (SEQ ID NO.: 30), containing HindIII and BamHI sites on their respective 5′ and 3′ ends). The oligos were mixed at 100 UV, heated, and amplified through one round of PCR (Amplitaq Gold®, Applied Biosystems) and then TOPO®-cloned into the pCR®4-TOPO® vector (Invitrogen). Inserts were sequenced and then sub-cloned into pcDNA4/myc-HisA (Invitrogen) to create pcDNA4/shKCNMA1.

2.5×10⁵ WM1552C or A375 melanoma cells were seeded into a single well of a 6-well plate and transfected overnight with 5 μg pcDNA4/miR-211, pcDNA4/shKCNMA1, or pcDNA4/myc-HisA (“vector only” negative control) using Fugene® 6 (Roche). The transfected cells were selected at 400 or 800 μg/mL Zeocin™ for 15 days, and the presence of the transgene copy in stable Zeocin™-resistant foci was confirmed by PCR (Amplitaq® Gold, Applied Biosystems). Cell lines were named WM1552C/211(400) or A375/211(400) when selection was at 400 μg/ml Zeocin™, and WM1552C/211(800) when selection was at 800 μg/ml Zeocin™, respectively. The “vector only” control cells were selected at 800 μg/ml Zeocin™. WM1552C/KC KO were selected at 400 μg/ml Zeocin™.

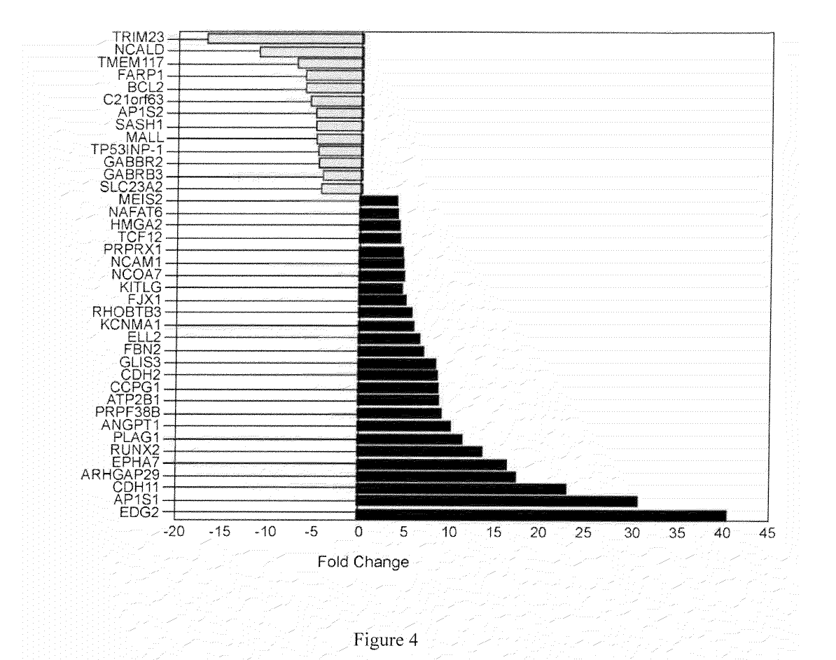

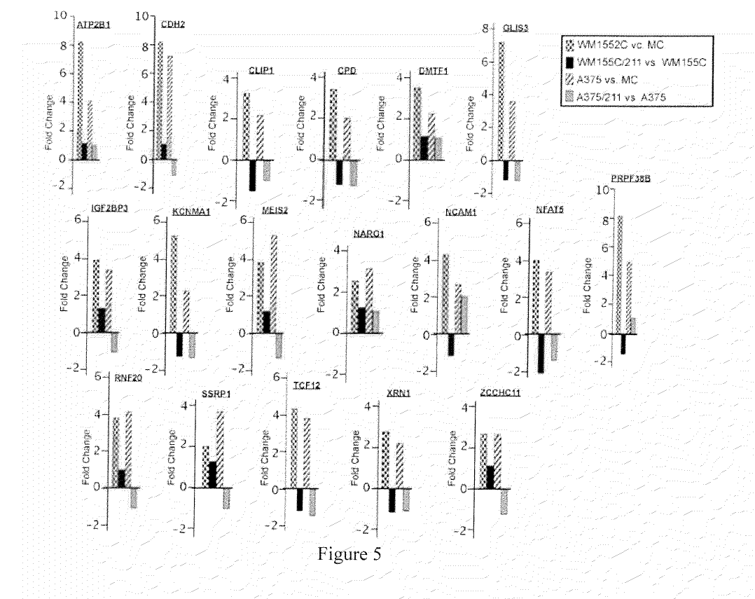

To demonstrate that depleted miRNA in melanoma is biologically relevant, (i.e., mechanistically related to melanoma development as opposed to coincidental) melanoma cells were assessed for enrichment in their target transcripts levels relative to their corresponding levels in melanocytes. As the first step to identify such mRNA transcripts, cDNAs made from total RNA isolated from the melanoma cell line WM1552C and the melanocyte line HEM-1 were hybridized to Affymetrix expression arrays. The hybridization intensity data was then filtered for differential expression of computationally predicted target transcripts of miR-211 (FIG. 4). These experiments revealed 26 putative target transcripts whose expression levels were elevated relative to those in HEM-1.

If the set of 26 genes contains valid targets of miR-211, the levels should be depleted if miR-211 levels increase in any melanoma cell line. To directly examine this possibility, three independent melanoma cell lines were constructed that stably express miR-211. For that purpose, the pre-miR-211 sequence (plasmid pcDNA4/miR-211) was transfected into WM1552C and A375 cells, followed by selection for stable expression of miR-211 and confirmation of expression by qRT-PCR analysis. The melanoma cell line clones that ectopically expressed miR-211 were named: WM1552C/211(400), WM1552C/211(800) and A375/211. Global mRNA levels in WM1552C/211(400) and A375/211 cells were measured on Affymetrix arrays and these levels were compared with the corresponding levels measured in the same experiment in untransfected parental cell lines WM1552C and A375, respectively. This analysis revealed a list of 18 putative target transcripts for miR-211, which were down-regulated by the artificial expression of miR-211 in both melanoma cell lines (FIG. 5). When cross-referenced with results reported in FIG. 4, nine of these putative target transcripts were found to be up-regulated in both melanoma cell lines compared to those in melanocytes and down-regulated in both melanoma cell lines when miR-211 was stably expressed. These candidate targets of miR-211 are: ATP2B1, CDH2, GLIS3, KCNMA1, MEIS2, NCAM-1, NF-AT5, PRPF38B, and TCF12. The expression of KCNMA1, which encodes a component of a K⁺ exporting channel whose function is modulated by Ca⁺⁺, has been linked to tumor cell proliferation in prostate cancer [49], cell migration in glioma [56] and antineoplastic drug resistance in melanoma cells [57]. The 3′-UTR of the KCNMA1 transcript also contains one of the strongest predicted target sites of miR-211. Therefore this transcript was the focus of further investigation.

### Example 4

### KCNMA1 Protein and Transcript Levels Correlate Inversely with that of miR-211

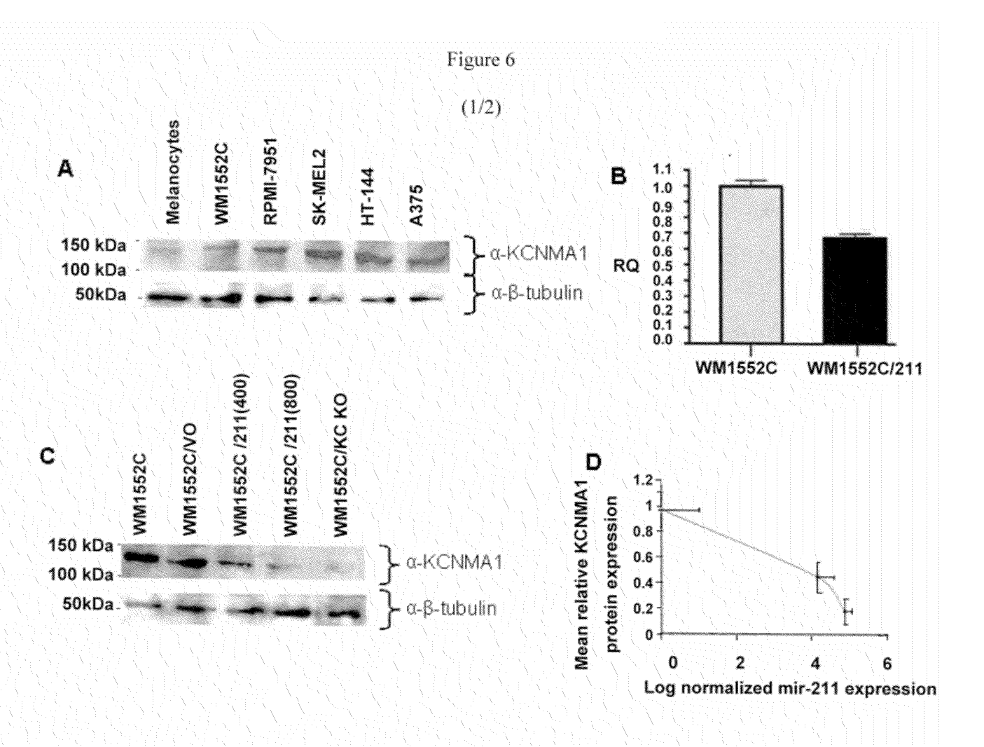

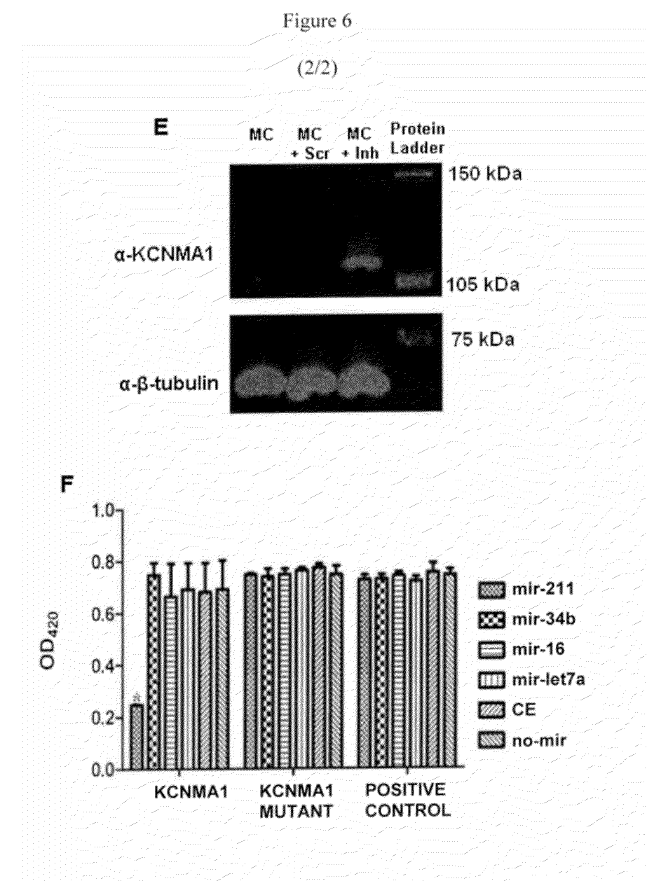

If miR-211 targets the KCNMA1 transcript, KCNMA1 protein expression levels should inversely correlate with that of miR-211 expression levels. A western blot analysis of KCNMA1 expression was performed, utilizing the same cell lines previously examined by northern blot (FIG. 2B) for KCNMA1 transcript expression. KCNMA1 protein expression was very low in normal melanocytes, but high in all melanoma cell lines (FIG. 6A), indicating an inverse correlation of expression between KCNMA1 protein and miR-211.

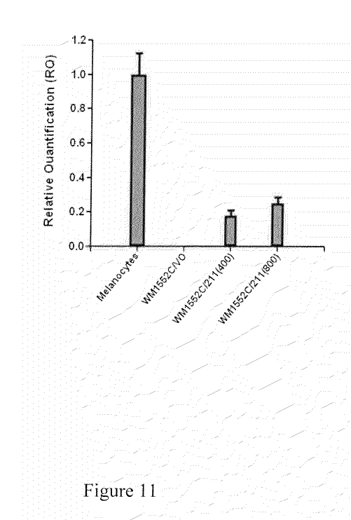

qRT-PCR analyses were then run to determine whether the induced expression of miR-211 in melanoma cells could reduce KCNMA1 transcript levels. KCNMA1 expression in wild type WM1552C was compared with that in WM1552C/211(400), revealing that the introduction of miR-211 down-regulates the KCNMA1 transcript (FIG. 6B). To further address whether KCNMA1 mRNA levels reflected KCNMA1 protein expression, a western blot analysis was performed looking for KCNMA1 in cell extracts obtained from: 1) WM1552C, 2) WM1552C/VO (WM1552C cells with a stably-incorporated empty expression vector), 3) WM1552C/211(400), 4) WM1552C/211(800), and 5) WM1552C/KC KO (WM1552C cells with a stably-expressing shRNA against the KCNMA1 mRNA) (FIG. 6C). It was found that KCNMA1 protein levels were significantly reduced in both melanoma cell lines expressing miR-211 [even more so in WM1552C/211(800)] compared to those in WM1552C/VO or untransfected WM1552C cells. KCNMA1 was virtually undetectable in the WM1552C/KC KO cell line. These results are consistent with the idea that miR-211 is able to target the KCNMA1 mRNA, thereby decreasing the amount of KCNMA1 protein in the cell. miR-211 expression was measured in engineered melanoma cell lines by qRT-PCR, and it did not exceed the levels observed in, melanocytes (FIG. 11). To further confirm our observations, we measured the correlation between miR-211 expression and KCNMA1 protein levels (FIG. 6D). The results revealed an inverted correlation between miR-211 expression and KCNMA1 protein levels. To confirm that this expression correlation occurred in non-transformed cells in addition to cancerous cell lines, the effect of miR-211 inhibition on the expression of KCNMA1 in melanocytes was examined. Melanocytes were transfected with anti-miR-211 inhibitors (Exiqon) and the protein expression of KCNMA1 was measured. The results indicated that derepression of KCNMA1 protein expression could be achieved by inhibition of miR-211 (FIG. 6E).

### Example 5

### miR-211 Directly Targets the KCNMA1 Transcript

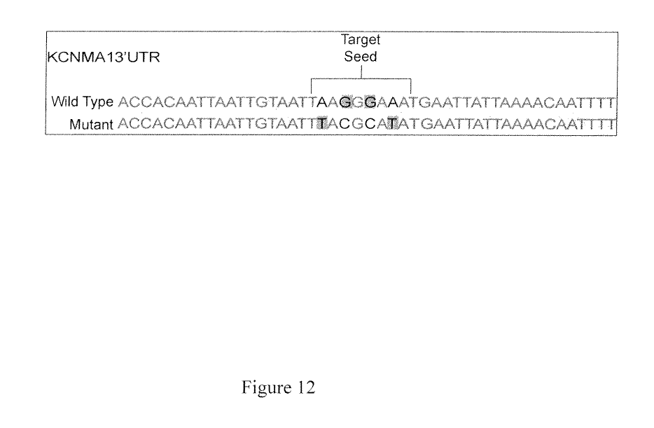

The 3′ UTR seed sequences of putative target genes were amplified by PCR (Phusion™ PCR kit, Finnzymes) from human melanocyte genomic DNA (Primers: KCNMA1 For—tgcggccgccttccctatatctaaacaatgcaaaatc (SEQ ID NO.: 31), KCNMA1 Rev—aaccggtcacccatccaggcgaggagc (SEQ ID NO.: 32), the primer set contained 5′ NotI or 3′ AgeI sites). The PCR product was cloned into pCR®4-TOPO® (Invitrogen), confirmed by sequencing, then sub-cloned into the 3′ UTR of the LacZ gene in pcDNA6/V5-His/LacZ (Invitrogen) using the 5′ NotI and 3′ AgeI restriction sites and reconfirmed by sequencing (pcDNA6/LacZ/KCNMA1). The cloned 3′UTR of KCNMA1 was mutated using the primers: KC Mut For—TACGCATATGAATTATTAAAACAATTTT (SEQ ID NO.: 33) and KC Mut Rev—TATGCGTAAATTACAATTAATTGTGCT (SEQ ID NO.: 34), and used to PCR amplify pcDNA6/LacZ/KCNMA1 using Quickchange (Stratagene). The plasmid product was then recovered and confirmed by sequencing (pcDNA6/LacZ/KCNMA1-MUT, see FIG. 12 for mutagenesis). A375 melanoma cell lines were then transfected in triplicate (Fugene® 6, Roche) with 5 μg plasmid DNA of: A) pcDNA6/LacZ/KCNMA1, B) pcDNA6/V5-His/KCNMA1-MUT or C) pcDNA6/V5-His/LacZ (positive control), and co-transfected (siPORT™, Ambion) at 100 nM with miRIDIAN microRNA Mimics (Dharmacon) for A) miR-16-1, B) miR-211, C) miR-34b, D) miR-let-7a-1, E) miRIDIAN cel-miR-67 (negative control: cel-miR-67 has been confirmed to have minimal sequence identity with miRNAs in human, mouse, and rat), or F) no mimic miRNA. After overnight incubation, cells were washed in PBS and reincubated in fresh media. After 48 hours, cells were harvested by trypsinization, examined for viability, and samples were prepared for the β-galactosidase assay using the β-Gal Assay kit (Invitrogen). Samples were incubated overnight at 37° C., then assayed for β-galactosidase activity in a 96-well plate format using a FlexStation3 (Molecular Devices).

To determine whether the computationally predicted target site of miR-211 in the 3′-UTR of the KCNMA1 transcript confers sensitivity to miR-211, a target cleavage assay was performed with a construct containing the 3′-UTR of KCNMA1 cDNA fused downstream of the reporter gene β-galactosidase. The construct, pcDNA6/LacZ/KCNMA1, as well as a derivative, pcDNA6/LacZ/KCNMA1-MUT (containing a mutated target cleavage site at the seed sequence; see FIG. S3), and the control vector pcDNA6/LacZ, were separately transfected into A375 cells along with one of the following miRNA mimics: miR-211, miR-16-1, miR-34b, miR-let-7a-1, cel-miR-67, or no mimic (FIG. 6E). The results revealed a statistically significant drop of nearly 60% in 3-galactosidase activity when the cells were transfected with pcDNA6/LacZ/KCNMA1 together with miR-211 mimics, but not with any other combination. Importantly, this drop was not detectable in cells co-transfected with pcDNA6/LacZ/KCNMA1-MUT and the miR-211 mimic, demonstrating that miR-211 was capable of specifically targeting the wild type seed sequence in the 3′-UTR of the KCNMA1 transcript.

### Example 6

### MITF Coordinately Regulates miR-211 and TRPM1

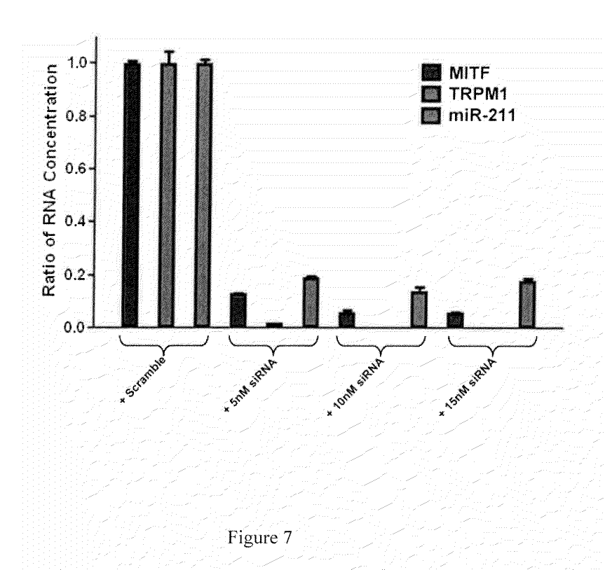

The gene encoding miR-211 is located within the sixth intron of the TRPM1 gene, which encodes multiple polypeptide isoforms including melastatin-1, a transient receptor potential (TRP) protein family member thought to be a potential suppressor of melanoma metastasis [58]. However, the molecular basis of the tumor suppressor activity of TRPM1 gene is not understood. The transcription factor MITF regulates the expression of TRPM1 gene, where the MITF-binding motif (GCTCACATGT) (SEQ ID NO.: 35) is located in the TRPM1 promoter [58]. In order to determine whether MITF also might transcriptionally regulate miR-211 expression via the TRPM1 promoter, it was determined that both TRPM1 and miR-211 transcripts are expressed in pigmented but not in the non-pigmented melanoma cells. To determine whether MITF expression modulates miR-211 expression, MITF expression was knocked down by siRNA in the pigmented melanoma cell line SK-MEL28. Three different doses of siRNA (5 nM, 10 nM and 15 nM) were used, and the knock-down efficiency was measured by qRT-PCR. As expected, the extent of reduction in MITF transcript levels directly correlated with the reduction in TRPM1 and miR-211 transcript levels (FIG. 7). These results suggest that MITF co-ordinately regulates TRPM1 and miR-211 expression. It also suggests that one of the ways MITF might also suppress melanoma metastasis is through its transcriptional activation of miR-211 via the TRPM1 promoter, and the consequent negative post-transcriptional effects of miR-211 on KCNMA1 mRNA.

### Example 7

### The Effect of miR-211 on Cell Proliferation and Invasion

### Proliferation

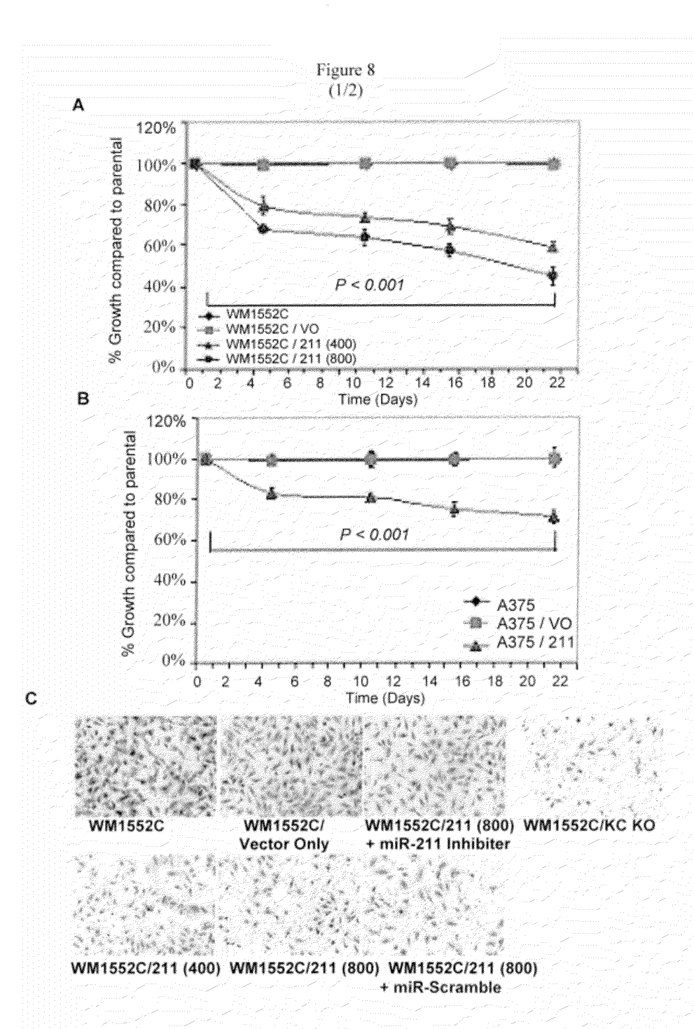

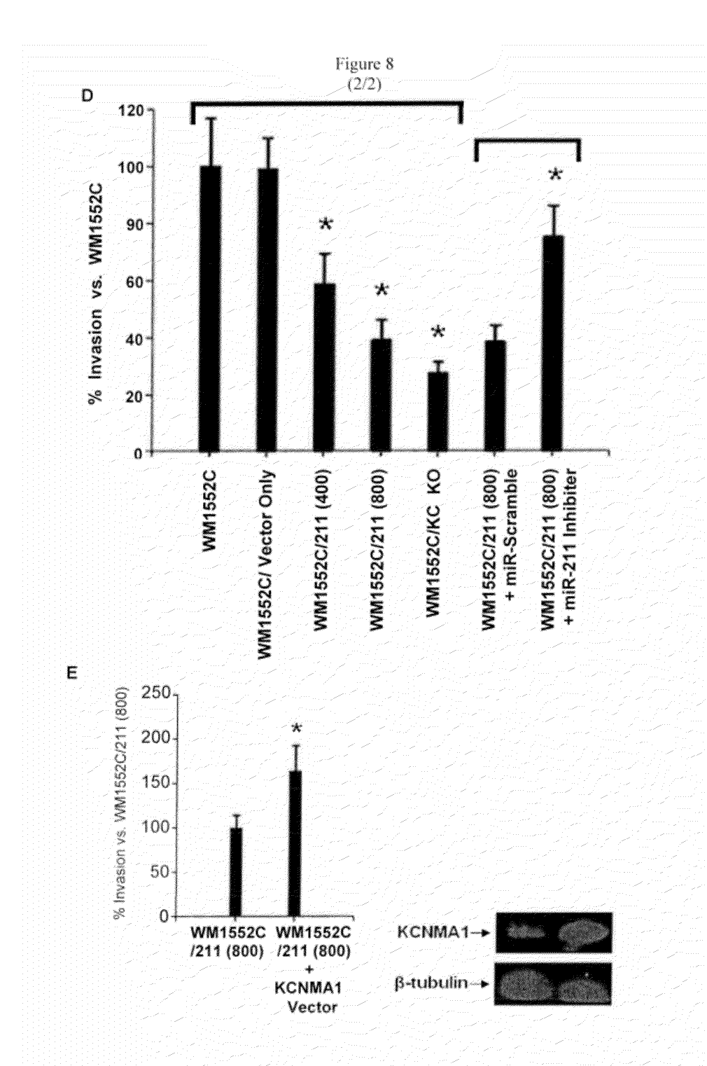

The over-expression of KCNMA1 is often associated with both cell proliferation and cell migration/invasion in various cancers [49-51]. Therefore, the effects of depletion of miR-211 and associated over-expression of KCNMA1 on these process in melanoma cells were determined. Proliferation rates of melanoma cell lines stably transfected with the miR-211 expression cassette were compared with those of untransfected melanoma cells and cell lines transfected with the empty expression vector (FIG. 8A), respectively. All miR-211-expressing cultures of WM1552C/211 showed reduced cell counts compared to those of WM1552C beginning at even the first time point (day 4), and the titer continued to fall behind as time progressed. After a 21-day period, WM1552C/211(400) had greater than 30% decrease in cell counts compared to those of WM1552C, while WM1552C/211(800) cultures showed an even greater decrease in cell proliferation. WM1552C/VO cells showed no significant difference in cell proliferation compared to WM1552C. Comparable results were obtained for cell proliferation of A375/211 cell lines, which grew more slowly than untransfected A375 or A375/VO (FIG. 8B). These results are consistent with the hypothesis that an important growth stimulatory event in the melanoma cell lines WM1552C and A375 involves the depletion of miR-211 levels—the latter possibly leading to the targeted up-regulation of at least KCNMA1/expression among its target genes.

### Invasion

Total lysates of 5×10⁵ cells of each cell line were boiled under denaturing conditions and proteins separated on 6% Tris-Glycine denaturing polyacrylamide gels by electrophoresis. Proteins transferred to nitrocellulose membranes were probed with the following primary antibodies: anti-Slo1 (NcuroMab, UC Davis) at 1/500 and anti-β-tubulin (BD Pharmingen) at 1/2000 according to standard methods. Blots were probed with horseradish peroxidase-conjugated secondary antibodies and visualized with ECL chemiluminescence (Pierce) or Alexa 680-conjugated secondary antibodies (Molecular Probes) and visualized on the Licor Odyssesy (Licor).

Assays were performed using WM1552C, WM1552C/V0, WM1552C/211(400), WM1552C/211(800), A375, A375/VO, and A375/211 cell lines. Cells were grown in log phase, trypsinized, counted using an automated cell counter (Cellometer®, Nexcelom Bioscience), and then seeded into 75 cm² flasks at 5×10⁵ cells per flask (in triplicate). Media was changed after 6 hours, and cells were further fed every 48 hours (Complete Tu Media). At days 4, 10, 15, and 21, cells were trypsinized, counted (Cellometer®, Nexcelom Bioscience), and then reseeded. Each assay was performed in duplicate for all cell lines.

BD BioCoat™ growth factor reduced insert plates (Matrigel™ Invasion Chamber 12 well plates) were prepared by rehydrating the BD Matrigel™ matrix coating in the inserts with 0.5 mls of serum-free Complete Tu media for two hours at 37° C. The rehydration solution was carefully removed from the inserts, 0.5 ml Complete Tu (2% FBS) was added to the lower wells of the plate, and 2.5×10⁴ cells suspended in 0.5 ml of serum-free Complete Tu media were added to each insert well. WM1552C/211(800) cells were additionally transfected with the Anti-miR miRNA Inhibiter for hsa-miR-211 as well as Negative Control#1 (Ambion) (miR-Scramble) at a concentration of 100 nM using siPORT NeoFX (Ambion). Invasion assay plates were incubated for 48 hours at 37° C. Following incubation, the non-invading cells were removed by scrubbing the upper surface of the insert. The cells on the lower surface of the insert were stained with crystal violet, and each trans-well membrane was mounted on a microscope slide for visualization and analysis. The slides were scanned using the Aperio Scanscope XT and visualized using the Aperio Imagescope v10 software. The number of migrating tumor cells was counted from each of five images per cell line (including miR Inhibiter transfected cells) in the central area of the filter. Cell lines were tested in triplicate, and the assays were performed twice. Data are expressed as the percent invasion through the membrane relative to the migration of WM1552C (Wild Type) through the membrane.

5×10⁵ HEM-1 cells were seeded into wells of a 6-well plate. The cells were then transfected with Fugene® 6 (Roche) and either 100 nM of anti-miR-211 Inhibitors (Exiqon), 100 nM of anti-miR Inhibiter Negative Control #1 (“miR-Scramble”), or transfection agent only. After 48 hours, the cells were harvested by trypsinization and counted using an automated cell counter (Cellometer®, Nexcelom Bioscience). 2.5×10⁵ cells were then prepared for western blotting (as above).

The impact of miR-211 expression on the invasive properties of WM1552C. WM1552C/211(400) and WM1552C/211(800) cells, along with WM1552C/VO, WM1552C/KC KO, and untransfected WM1552C was determined. Cells were seeded separately into invasion chambers, and the cells were allowed to migrate as described above. Results indicated that WM1552C/211(400) and WM11552C/211(800) cells migrated significantly less (˜40% and 60% less, respectively) than WM1552C (FIGS. 5C and 8D), whereas WM1552C/VO cells showed almost no variation compared to parental cells. The frequency of cells with invasion defects significantly exceeded the decrease in the proliferation rates of these cells (an ˜8-10% decrease in growth over the 48 hours of the invasion assay period), suggesting that the two effects on miR-211 expression are independent of each other. The most significant effect on invasion was observed in the WM1552C/KC KO cells. While a sequence-scrambled oligonucleotide (miR-Scramble) did not show an effect on cell invasion, cells treated with a miR-211 inhibitor restored the invasion phenotype by as much as 40% (FIG. 8D). Given that previously published evidence directly links KCNMA1 gene dosage and/or expression with increased motility/invasion in several cancers [49-51], these results suggest that at least part of the invasion defect caused by miR-211 in melanoma cell lines is due to targeted down-regulation of the KCNMA1 transcript.

### Effect of KCNMA1 on Proliferative and Invasive Functions

2.5×10⁵ cells WM1552C/211(800) cells were seeded into wells of a 6-well plate. 1 well was transfected with 5 μg of KCNMA1-expressing plasmid (Origene catalog SC122078) using Fugene® 6 (Roche) and a second well was treated with transfection reagent only. After 48 hours, the cells were harvested by trypsinization and counted using an automated cell counter (Cellometer®, Nexcelom Bioscience). 2.5×10⁴ cells were then utilized for invasion assays (in triplicate) and 2.5×10⁵ cells were prepared for western blotting (as above).

To fully demonstrate that KCNMA1 is a key contributor to miR-211 effects, we examined whether concomitant over-expression of KCNMA1 might also rescue the miR-211 anti-invasive effects. A KCNMA1 constitutively-expressing plasmid was transiently transfected into WM1552C/211(800) cells. This plasmid (Origene clone NM_{—}002247.2) contains a KCNMA1 ORF without its native 3′UTR (making it resistant to regulation by miR-211). KCNMA1 protein expression levels were then detected by KCNMA1 antibody. Western blot results revealed that KCNMA1 protein levels were elevated in transfected cells [“WM1552C/211(800)+KCNMA1 vector” relative to control cells] (FIG. 8E, bottom). Results from an invasion assay (FIG. 8E, top) illustrate that the same batch of melanoma cells that exhibit high KCNMA1 protein expression [WM1552C/211(800)+KCNMA1 vector” cells] also show high cell invasiveness, higher by at least 60% compared to the control cell cultures.

### Example 8

### Mutation in TRPM1 Promoter Down-Regulates Expression of miR-211

### Sequencing of Upstream TRPM1 Promoter

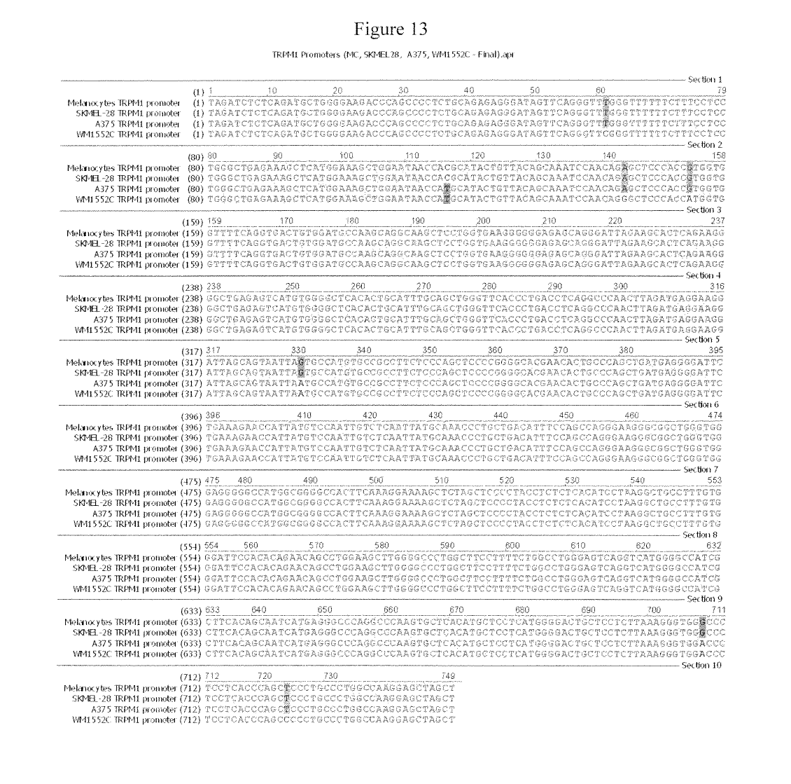

In order to determine whether the differences in miR-211 expression between melanocytes and invasive melanoma result from differences in the expression of the TRPM1 gene, the TRPM1 gene promoter analyzed. Sequencing alignment and comparison was performed for the upstream TRPM1 promoter region of melanocytes and three cell lines: SKMEL-28, A375, and WM1552C. The promoter sequences from each cell type are shown in FIG. 13. The alignment revealed that the upstream TRPM1 promoter region of TRPM1 is identical between melanocytes and SKMEL-28 cells. However, point mutations were detected in both the A375 and WM1552C cell lines, three of which are identical between the two cell lines. The data demonstrates that SKMEL-28 cells express miR-211 because the TRPM1 gene is under the control of a wild-type in promoter; whereas both A375 and WM1552C cells, which do not express miR-211, have multiple point mutations in their TRPM1 promoters.

### Luciferase Reporter Expression Assay

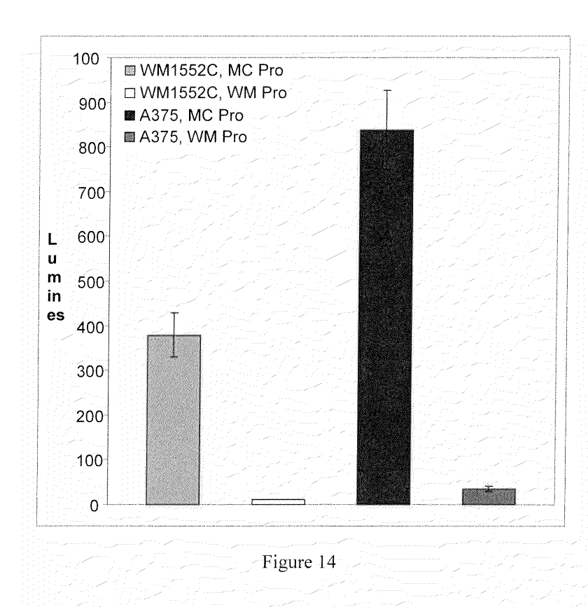

In order to investigate whether the mutations in the TRPM1 gene promoter of A375 and WM1552C cells affects (downregulates) the expression of the TRPM1 gene and miR-211, the luciferase reporter gene placed under operational control of either the melanocyte TRPM1 promoter (wildtype; “MC Pro”) or the WM1552C TRPM1 promoter (mutated promoter; “WM Pro”). Each of these constructs was transfected into WM1552C and A375 cells and luciferase luminescence was measured. In both cell lines, the melanocyte TRPM1 promoter is significantly more functional than the WM1552C TRPM1 promoter (FIG. 14). The results demonstrate that the point mutations present in the WM1552C TRPM1 promoter reduce the expression of TRPM1 and miR-211 regardless of cell type background.

### Example 9

### Effects of Treatment of SKMEL-28 Cells with MITF siRNAs

### Down-Regulation of MITF

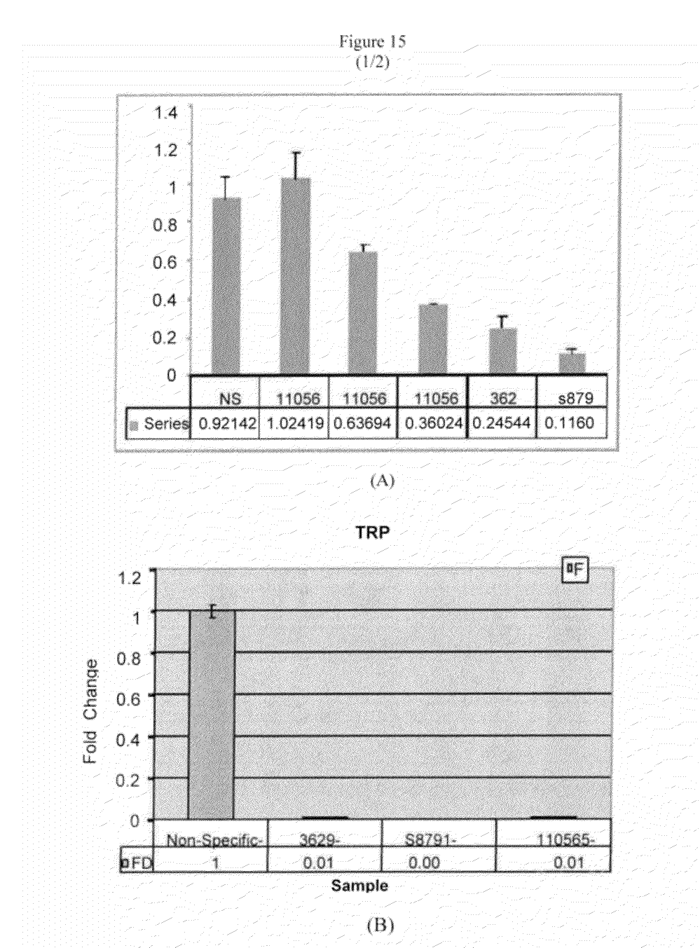

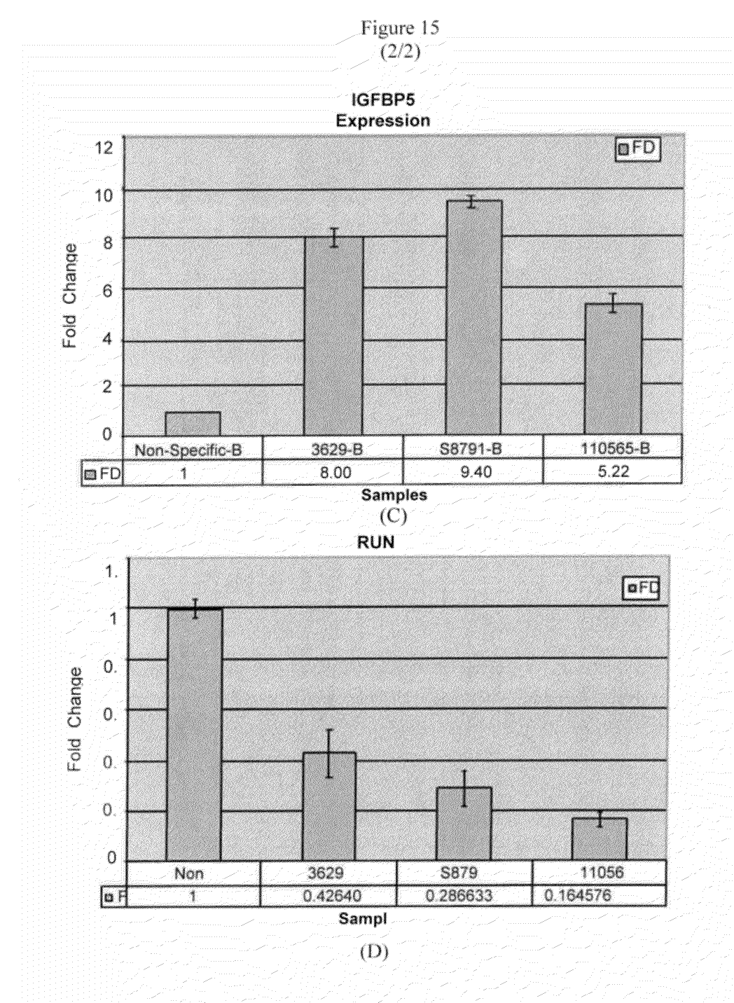

The effect of MITF down regulation on TRPM1 gene expression and downstream targets of miR-211 was assessed. SKMEL-28 cells were treated with either a nonsense miRNA or one of five different siRNAs specific to MITF: 110566, 110564, 110565, 3629, and s8791. Each of the five MITE siRNAs is a product ID of a Silencer® Select siRNA (Ambion, Applied Biosystems) for a validated siRNA. The sequences are proprietary, but map approximately to the 10th, 10th, 9th, 3rd, and 6th exons, respectively. The nonspecific (NS) control siRNA is a mix of 48 different non-specific siRNAs (Ambion) pooled together. Expression of MITF was determined for each group using qPCR detection methodology. The results, which are shown in FIG. 15A, demonstrate that four of the siRNAs (110564, 110565, 3629, and s8791) significantly down-regulated MITF (35%, 64%, 75%, and 90%, respectively).

### Down-Regulation of TRPM1

Expression of TRPM1 was determined via qPCR for SKMEL-28 cells treated with the three top-performing siRNAs as determined by the MITF down-regulation study described above. The results shown in FIG. 15B demonstrate that the three siRNAs tested (110565, 3629, and s8791) significantly down-regulated TRPM1. This confirms that MITF acts as a transcription factor to positively-upregulate TRPM1, since a knock-down of MITF by the three siRNAs resulted in significant down-regulation of TRPM1, and, in the case of s8791, a complete silencing of TRPM1. As discussed above, it is expected that down-regulation of TRPM1 results in a down-regulation of miR-211 expression.

### Up-Regulation of IGFBP5

Expression of IGFBP5, a target of miR-211, was assessed using qPCR for SKMEL-28 cells treated with the three best-performing siRNAs as determined by the MITF down-regulation study. With the lack of TRPM1 expression due to MITF knock-down, IGFBP5 is up-regulated 8-fold for siRNA 3629, 9.4-fold for s8791, and 5.22-fold for 110565 (FIG. 15C).

### Down-Regulation of RUNX2

RUNX2 is a putative target of miR-211 and therefore would be expected to be up-regulated following MITF knock-down. However, as shown in FIG. 15D, RUNX2 is consistently down-regulated in response to MITF knock-down. This finding suggests that RUNX2 is positively dependant upon MITF expression and therefore is unlikely to be a miR-211 target.

### Example 10

### IGFBP5 mRNA is Down-regulated by miR-211 Expression

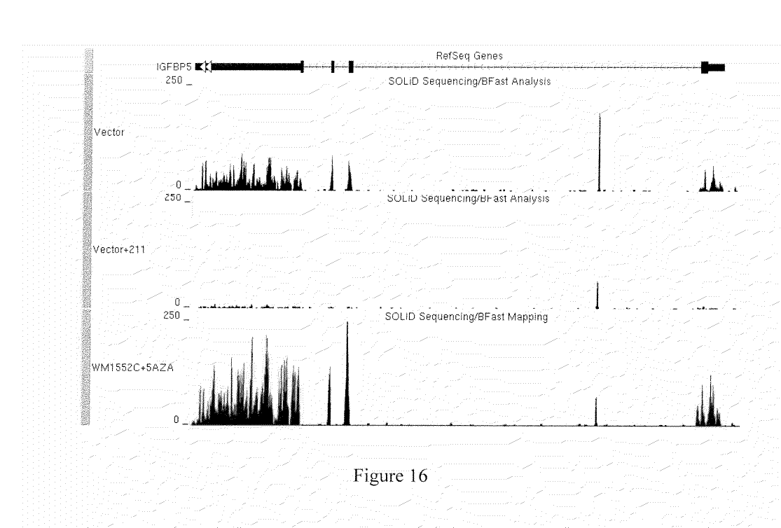

To extend the findings of the MITF knock-down study and to further establish IGFBP5 as a miR-211 target, the effect of miR-211 expression on IGFBP5 mRNA was determined. WM1552C cells were transfected with either an empty vector or the vector encoding and expressing miR-211. Additionally, untransfected WM1552C cells were treated with 5-Aza-2′ deoxycytidine (“5-Aza”) to investigate whether miR-211 down-regulates IGFBP5 mRNA production through a genomic methylation mechanism. The sequencing results shown in FIG. 16 demonstrate that IGFBP5 mRNA is expressed in the IGFBP5 locus in WM1552C cells. However, over-expression of miR-211 reduces IGFBP5 mRNA expression to almost to undetectably levels, thereby validating IGFBP5 as a target of miR-211. 5-Aza treatment had no effect on the expression of IGFBP5 mRNA, suggesting that DNA methylation is not a means by which IGFPB5 is down-regulated by miR-211.

### Example 11

### Target Inhibition Assay of IGFBP5 3′UTR by miR-211 Using Luciferase Reporter Assay

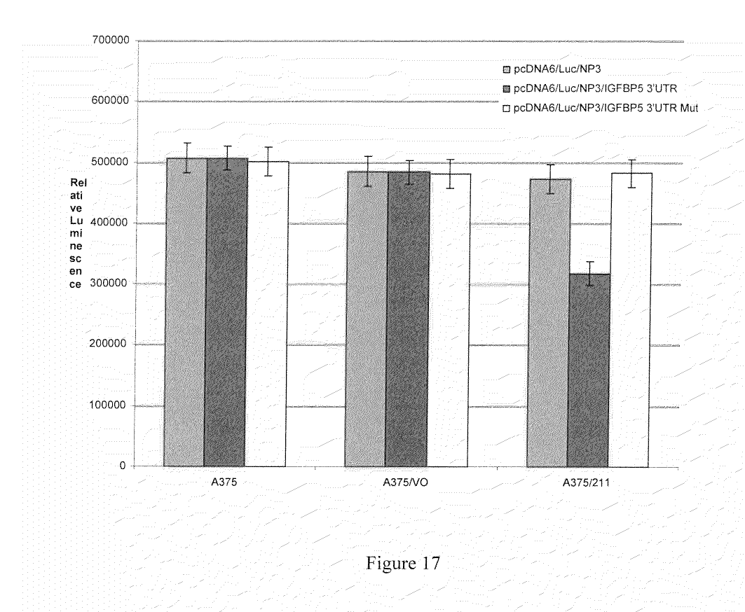

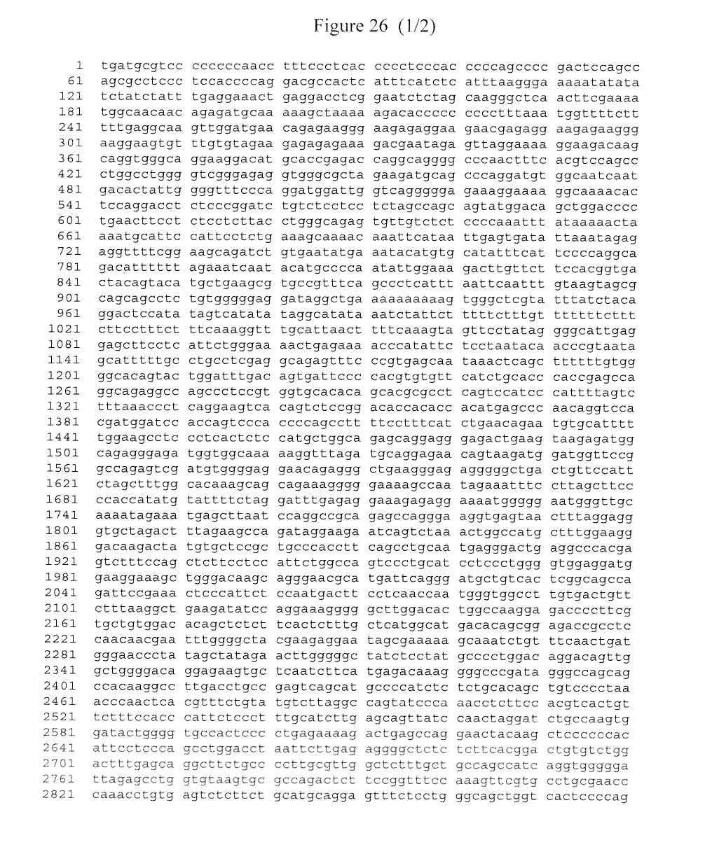

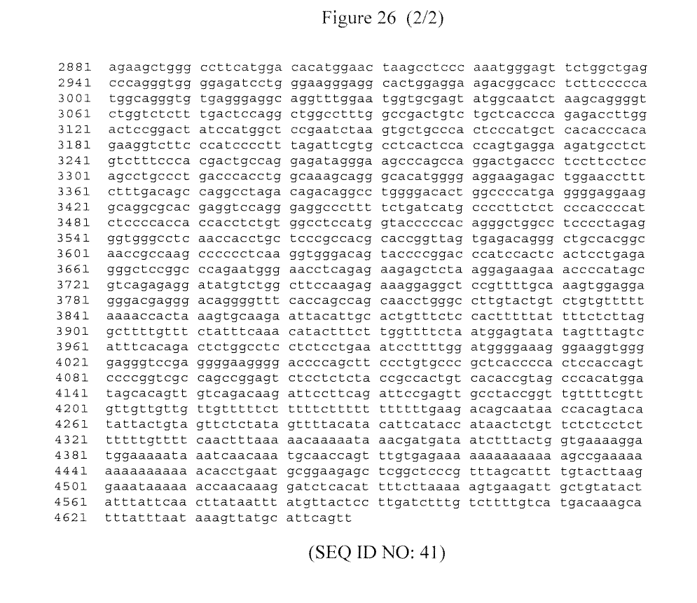

A putative miR-211 binding site with the sequence 5′-aaagggaa-3′ (SEQ ID NO:40) is present in the 3′-UTR of the IGFBP5 mRNA (SEQ ID NO:41; FIG. 26). In order to investigate the specificity and mechanism of miR-211 inhibition of IGFBP5, three luciferase reporter constructs were created, each having a different 3′-UTR on the luciferase gene. The constructs were as follows: (i) luciferase coding sequence with the luciferase (“pcDNA6/Luc/NP3”), (ii) luciferase coding sequence with the IGFBP5 3′-UTR (“pcDNA6/Luc/NP3/IGFBP5 3′UTR”), and (iii) luciferase coding sequence with the IGFBP5 3′-UTR having a mutation in the miR-211 binding site (“pcDNA6/Luc/NP3/IGFBP5 3′UTR Mut”). The mutant miR-211 binding site is represented by SEQ ID NO:42 (5′-taagccta-3′). Vectors encoding these constructs were transfected into native A375 cells, A375 cells containing the miR-211-expressing vector (“A375/211”), or A375 cells having an empty vector (“A375/VO”). As shown in FIG. 17, expression of luciferase is strong in native A375 cells regardless of the 3′UTR present in the reporter plasmid (either native luciferase, wildtype IGFBP5 3′UTR, or mutated IGFBP5 3′UTR). Expression of luciferase was also indistinguishable in the A375/VO (vector only) cells which do not express significant levels of miR-211. In the miR-211-expressing A375 cells, luciferase expression was unaffected for the constructs containing the native luciferase 3′UTR and the IGFBP5 3′UTR in which the miR-211 binding site had been altered. However, the luciferase activity was reduced by nearly 40% when the native IGFBP5 3′UTR was expressed. These results confirm that IGFBP5 is a target of miR-211 and that miR-211 reduces IGFBP5 expression by its action at the 3′UTR.

### Example 12

### Effects of TP53 siRNAs on MITF, TRPM1, and IGFBP5

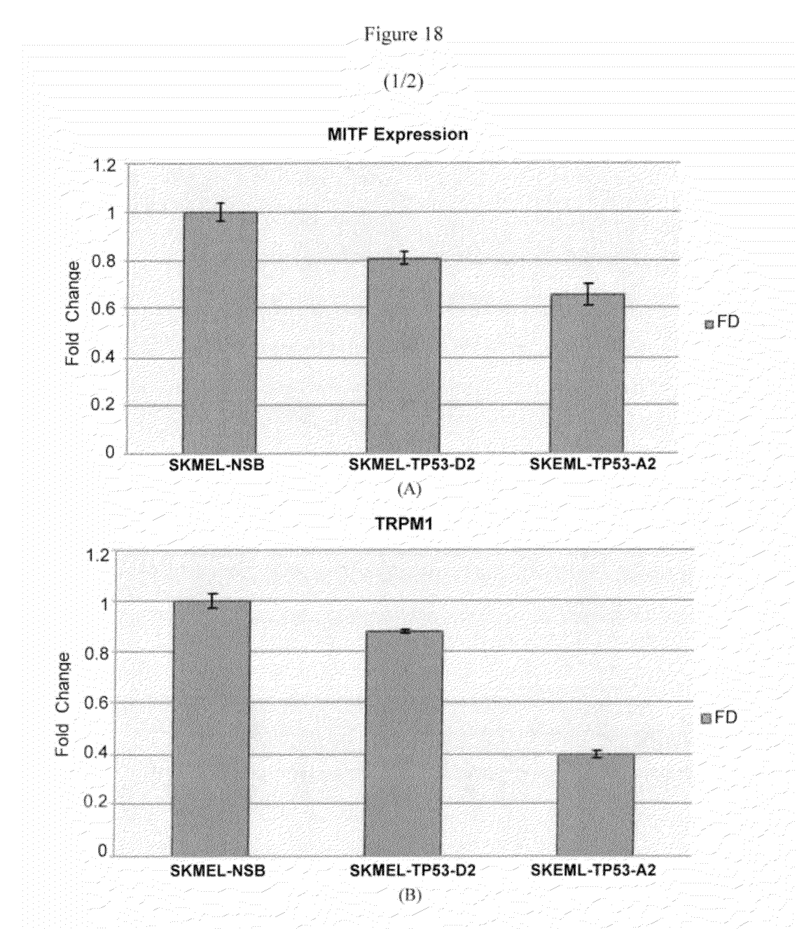

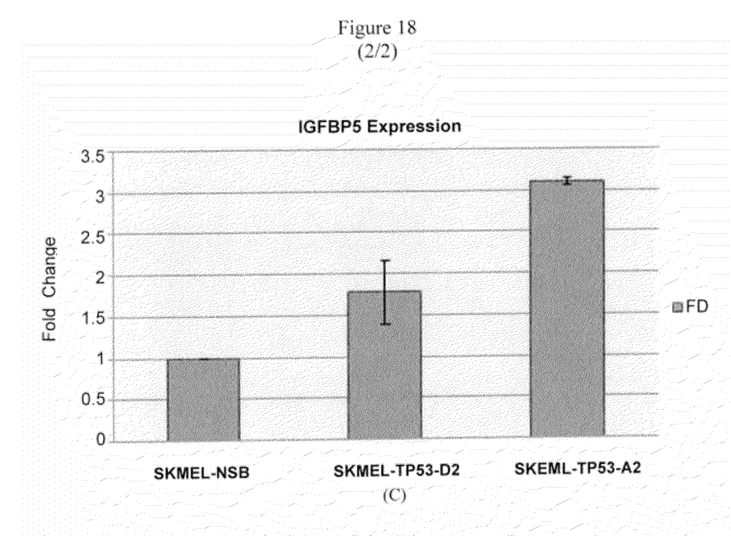

The role of TP53 (a putative upstream effector of MITF) was investigated. Four different TP53 siRNAs were transfected into SKMEL-28 cells for 48 hours. RNA was purified, and qRT-PCR was performed, normalized to GAPDH. Two siRNAs (TP53-A2 and TP53-D2, proprietary Silencer® siRNAs with Ambion/Applied Biosystems product IDs 106141 and 2533, respectively, and which map approximately to the 11th and 6th exons, respectively, of TP53) induced a down-regulation of TP53 by greater than 90%. These siRNAs were then tested for downstream effects on MITF, TRPM1, and IGFBP5 expression. RNA samples were acquired, and qPCR was performed using Taqman probes. FIG. 18A-C, demonstrate that TP53 knockdown resulted in significant reductions in the expression of MITF, TRPM1 mRNA. Consistent with the MITF/TRPM1/IGFBP5 pathway established in the previous experiments, IGFBP5 expression was markedly increased. The TP53-A2 siRNA was consistently more effective than the TP53-D2 siRNA.

### Example 13

### Effects of Hypoxic Conditions (Simulated and Actual) on Cell Lines with and without miR-211 Expression

### A375 and 552C Cells

A375 cells and WM1552C, both wild-type and miR-211-expressing, were subjected to treatment with 0, 250 nM, or 400 nM defroxamine (DFO) to simulate hypoxic conditions in order to determine whether miR-211 expression is capable of being regulated by changes in O₂ concentrations. Cell counts were performed for each treatment group.

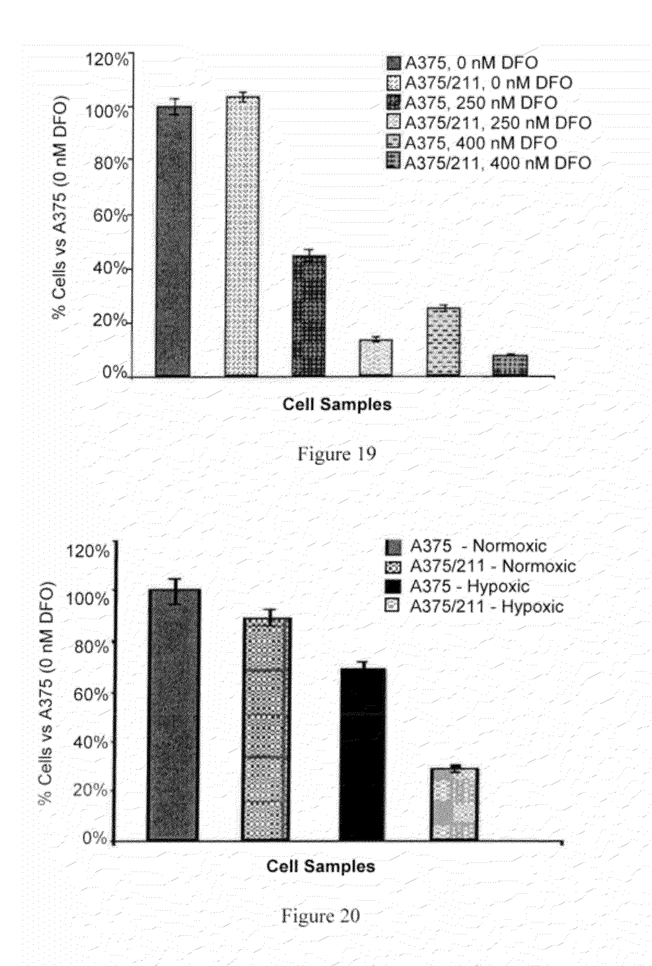

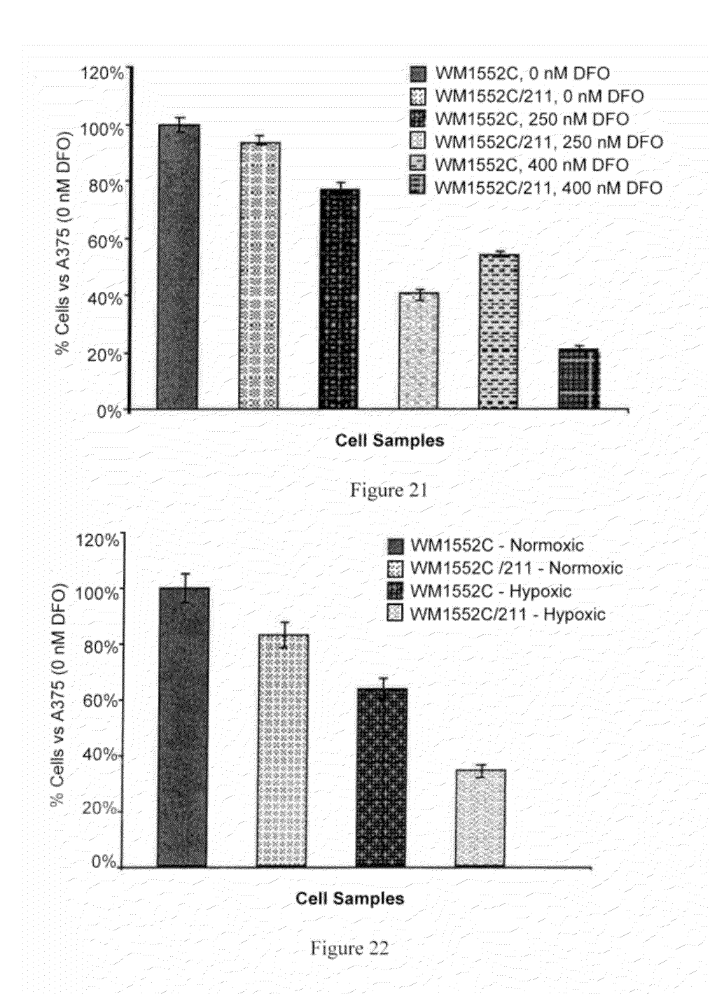

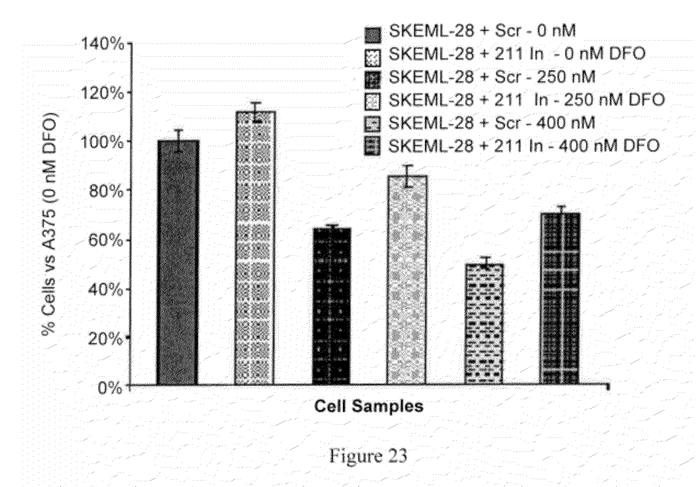

FIG. 19 shows the cell counts following DFO treatment in A375 cells. Relative to untreated cells, survival was about 45% following 250 nM DFO and about 25% following 400 nM DFO. The presence of miR-211 in these cells caused this effect to be greatly exacerbated, reducing survival to about 13% at 250 nM DFO and about 8% at 400 nM DFO. Similarly, for WM1552C cells, survival was about 77% at 250 nM DFO and about 55% at 400 nM DFO, compared to untreated cells (FIG. 21). The presence of miR-211 in WM1552C cells also resulted in increased cell loss with survival being about 40% at 250 nM DFO and about 21% at 400 nM DFO.

To test under actual hypoxic conditions, both cell lines were placed into a hypoxic chamber containing 2% O₂ prior to determination of cell counts. When compared to normoxic conditions for both A375 and A375/211 groups, the cell counts demonstrate that survival of A375 cells was about 68% of normoxic condition cells (FIG. 20). As in the DFO-simulated hypoxic condition assay, the presence of miR-211 caused the effect to be greatly exacerbated, reducing survival to about 29%. Likewise, hypoxic conditions for WM1552C cells resulted in about 64% survival which was reduced to 34% survival in cells expressing miR-211 (FIG. 22).

### SKMEL-28 Cells

SKMEL-28 cells, were subjected to the same DFO or hypoxic conditions as described above either in the presence or absence of an miR-211 inhibitor (has-miR-211 Anti-miR™ miRNA Inhibitor, Ambion, catalog numberAM17000, ID AM10168). DFO treatment resulted in a loss of SKMEL-28 cells, but not to the same extent as observed for the A375 and WM1552C cell lines. The survival of SKMEL-28 cells was about 64% at 250 nM DFO and about 50% 400 nM DFO, compared to untreated cells. Simultaneous treatment with the miR-211 inhibitor caused this deleterious effect to be somewhat rescued. The survival of the SKMEL-28 cells treated with the miR-211 inhibitor was about 85% at 250 nM DFO and about 70% at 400 nM DFO. Since SKMEL-28 cells express miR-211 highly, this indicates that the presence of miR-211 in wild-type cells is actually a hindrance to cell growth under hypoxic conditions.

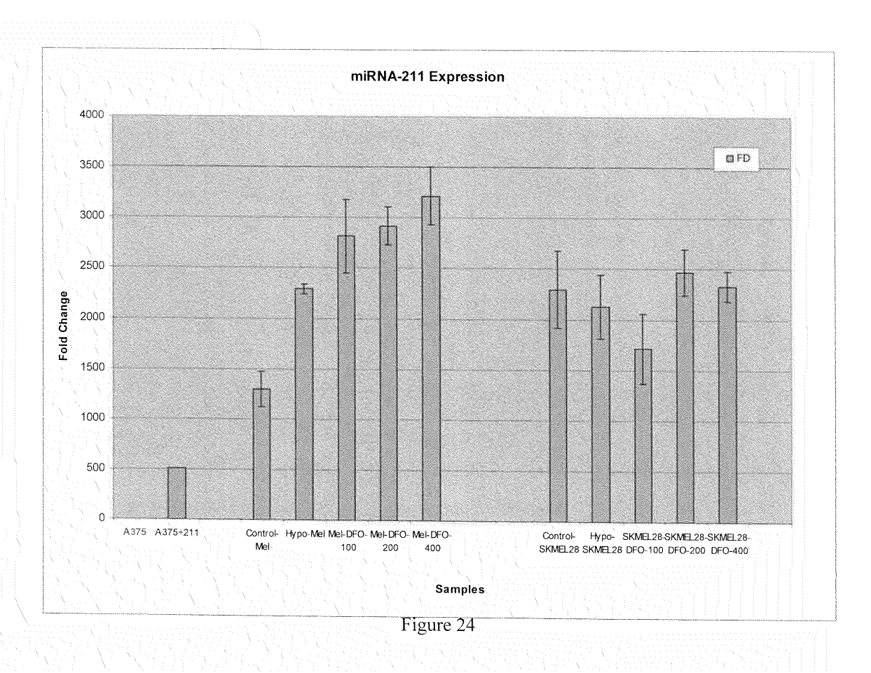

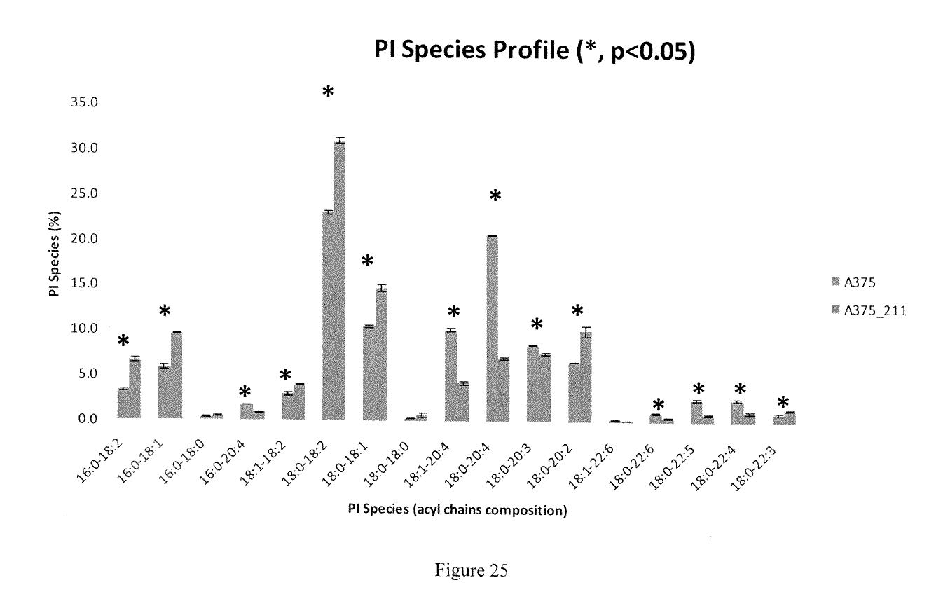

The effect of hypoxia on miR-211 expression was investigated under simulated (DFO) and actual hypoxic conditions in both melanocytes and SKMEL-28 cells. miRNA-211 expression was determined by qPCR. The results in FIG. 24 demonstrate that the expression of miR-211 is high in melanocytes and virtually undetectable in A375 cells. Melanocyte miR-211 expression is also significantly greater that miR-211 expression in the A375/211 cells. When hypoxia is induced in melanocytes, either by actual hypoxia (2% O₂) or using DFO, miR-211 expression is markedly increased. For DFO, miR-211 expression increases in a dose-dependent manner. However, neither simulated nor actual hypoxic conditions had a significant effect on miR-211 expression in SKMEL-28 cells, suggesting that miR-211 expression is not capable of being regulated by changes in O₂ concentrations in these melanoma cells.

### Example 14

### Effect of miR-211 Expression in A375 Cells on the Production of Lipid Species

To determine the effect of miR-211 expression in A375 cells on the production of lipid species, as an indicator of metabolic change, fatty acids were isolated and quantitated in A375 cells and A375/211 by mass spectrometry. The results, which are shown in a bar graph in FIG. 25, demonstrate that the presence of miR-211 is capable of altering the profile of lipid species produced in these melanoma cells. The most notable is the large increase in 18:0-18:2 acyl chains and the large decrease in content of 18:0-20:4 acyl chains in the A375/211 cells. This confirms that miR-211 is capable of altering the metabolic profile of these metabolic cells.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for diagnosing melanoma in a subject suspected of having melanoma comprising:
(i) assessing the expression level of miR-211 in a biological sample obtained from the subject;
(ii) comparing the expression level of miR-211 in the sample to the a reference expression level derived from the expression level of miR-211 in samples obtained from subjects diagnosed as not having melanoma; and
(iii) identifying the subject as having melanoma when the expression level of miR-211 in the sample is less than the reference expression level or identifying the subject as not having melanoma when the expression level of miR-211 in the sample is not less than the reference expression level.

2. The method of claim 1, wherein the biological sample comprises skin.

3. The method of claim 2, wherein the biological sample comprises skin epidermis.

4. The method of claim 3, wherein the biological sample comprises melanocytes.

5. The method of claim 1, wherein the expression level of miR-211 is assessed by evaluating the amount of miR-211 mRNA in the biological sample.

6. The method of claim 5, wherein evaluating the miR-211 mRNA comprises reverse transcriptase PCR (RT-PCR).

7. The method of claim 5, wherein evaluating the miR-211 mRNA comprises array hybridization, wherein the array comprises an immobilized nucleic acid probe that specifically hybridizes miR-211 mRNA, miR-211 cDNA, or complements thereof.

8. A method for determining the metastatic potential of melanoma in a subject comprising:
(i) assessing the expression level of miR-211 in a melanoma sample obtained from the subject; and
(ii) identifying the metastatic potential of the melanoma, wherein a lower expression level of miR-211 in the sample indicates a greater metastatic potential and a higher expression level of miR-211 in the sample indicates a lower metastatic potential.

9. The method of claim 8, wherein the expression level of miR-211 is assessed by evaluating the amount of miR-211 mRNA in the melanoma sample.

10. The method of claim 9, wherein evaluating the miR-211 mRNA comprises reverse transcriptase PCR (RT-PCR).

11. The method of claim 9, wherein evaluating the miR-211 mRNA comprises array hybridization, wherein the array comprises an immobilized nucleic acid probe that specifically hybridizes miR-211 mRNA, miR-211 cDNA, or complements thereof.

12. A method for determining the risk of a subject for developing melanoma comprising:
(i) assessing the expression level of TRPM1 in a biological sample obtained from the subject;
(ii) comparing the expression level of TRPM1 in the sample to the a reference expression level derived from the expression level of TRPM1 in samples obtained from subjects diagnosed as not having melanoma; and
(iii) identifying the subject as having increased risk of developing melanoma when the expression level of TRPM1 in the sample is less than the reference expression level or identifying the subject as not having an increased risk of melanoma when the expression level of TRPM1 in the sample is not less than the reference expression level.

13. The method of claim 12, wherein the biological sample comprises skin.

14. The method of claim 13 wherein the biological sample comprises skin epidermis.

15. The method of claim 13, wherein the biological sample comprises melanocytes.

16. The method of claim 12, wherein the expression level of TRPM1 is assessed by evaluating the amount of TRPM1 mRNA in the biological sample.

17. The method of claim 16, wherein evaluating the TRPM1 mRNA comprises reverse transcriptase PCR (RT-PCR).

18. The method of claim 16, wherein evaluating the TRPM1 mRNA comprises array hybridization, wherein the array comprises an immobilized nucleic acid probe that specifically hybridizes TRPM1 mRNA, TRPM1 cDNA, or complements thereof.

19. The method of claim 12, wherein the expression level of TRPM1 is assessed by evaluating the amount of TRPM1 protein in the biological sample.

20. A method for diagnosing a human as having melanoma or having an increased likelihood of developing melanoma, said method comprising,
(i) determining, in a sample obtained from a human, the presence or absence of a TRPM1 gene promoter mutation that causes a reduction in the TRPM1 gene expression relative to the TRPM1 gene expression from a TRPM1 gene promoter lacking that mutation, and
(ii) identifying the human has having melanoma or an increased likelihood of melanoma when a TRPM1 gene promoter mutation is identified, and identifying the human as not having melanoma or an increased likelihood of melanoma when the TRPM1 gene promoter mutation is absent.

21. The method of claim 20, wherein the TRPM1 gene promoter mutation is selected from the group consisting of T61C, C116T, A143G, A153G, G331A, G708A, and T724C mutations relative to SEQ ID NO: 36.

22. The method of claim 20, wherein the sample comprises skin.

23. The method of claim 21, wherein the biological sample comprises skin epidermis.

24. The method of claim 21, wherein the biological sample comprises melanocytes.
